(19) United States
(12) Patent Application Publication
(10) Pub. No.: US 2004/0106131 A1
(43) Pub. Date: Jun. 3, 2004

(54) COMPOSITIONS AND METHODS FOR PROTEOMIC INVESTIGATIONS

(76) Inventors: Swapan Roy, East Brunswick, NJ (US); John Krupey, Glen Rock, NJ (US); Matthew Kuruc, Englewood, NJ (US)

Correspondence Address: PROTEUS PATENT PRACTICE LLC, P.O. BOX 1867, NEW HAVEN, CT, 06508, US

(21) Appl. No.: 10/642,776

(22) Filed: Aug. 18, 2003

(51) Int. Cl. C12Q 1/68

(52) U.S. Cl. 435/6

(57) ABSTRACT

### Abstract of the Disclosure

The present invention provides a variety of related proteomics analytical modalities that are open-ended, rapid, convenient and suitable for implementation in a high throughput parallel assay system. Specificity-determining compositions and methods are disclosed for use in proteomics. These compositions and methods provide a protein resolved from other protein species contained in a sample fluid, in its native, biologically functional conformation. The present invention provides a specificity-determining substrate that forms a complex with a protein molecule in a homogenous fashion. The specificity-determining substrate includes a specificity-determining ligand bound to a support, wherein optionally the substrate further includes a spacer bound between the ligand and the support. In addition a complex is provided that includes a specificity-determining substrate and a protein molecule. Furthermore, an array including a plurality of loci is provided, in which each locus includes a specificity-determining substrate of the invention. These substrates, complexes and arrays may be employed in a method of resolving a first protein from a fluid including one or more species of native, biologically active protein molecules, wherein the first protein retains its native structure and its biological activity; in a method of purifying one or more first proteins from a fluid including one or more species of native, biologically active protein molecules, wherein the purified first protein retains its native structure and its biological activity; in a method of characterizing one or more proteins in a fluid including one or more species of protein molecule; and in a method of identifying one or more proteins in a sample fluid wherein the concentration of the one or more proteins in the sample fluid differs from the concentration of the one or more proteins in a reference fluid.

### Detailed Description of the Invention

### Cross Reference to Related Applications

This application claims the benefit of priority of Provisional Application U. S. Ser. No. 60/403,747 filed August 16, 2002, whose contents are incorporated herein in their entirety.

### Background of Invention

FIELD OF THE INVENTION

The present invention relates to compositions and methods for high specificity resolution of proteins from a mixture. More specifically, the invention relates to supports coupled to specificity-determining ligands, and to the methods of using them in proteomics research.

BACKGROUND OF THE INVENTION

As a result of the rapid advances in genomics the predicted amino acid sequences of tens of thousands of protein gene products for a given organism are now known. There is great interest for progress in treatment of human diseases, and more broadly, in fields such as agricultural genomics, to annotate these gene products. Many of the predicted proteins are unknown and others appear only weakly homologous or orthologous to other known proteins.

One objective of proteomic investigations is to provide annotations for proteins present in a cell, tissue or organ. There may exist several-fold gene products in a given cell originating from a given identified genomic sequence. This complexity may arise in many ways, including tissue- or organ-dependent alternative splicing of heteronuclear RNA prior to translation, post-translational modifications and processing in response to changes in cellular or tissue milieu. Furthermore, translational expression and post-translational modifications respond to changes in pathological conditions, such that a given protein species, including particular modifications may be present to different extents in a disease state than in a nondisease state, for the same tissue or organ. There is therefore a pressing need to provide proteomic analytical and diagnostic methodologies.

Current proteomic investigations are severely hampered by broad limitations of current data acquisition technology: resolution of protein content, automation compatibility, and the economics required of a high throughput system. Functional proteomic efforts to model and develop therapeutics, which generally require industrial-scale throughput and precision, exacerbate the problems of exact, rapid, massive and economical data acquisition. This makes protein separation and resolution fundamentally the critical barrier to industrial proteomics.

Two dimensional electrophoresis (2DE) is the predominant separation technique used today in proteomic analysis. Nevertheless 2DE suffers significant liabilities. First, there is a limitation of resolution. Analysis is done with respect to only two protein characteristics, size and isoelectric point. Resolution is compromised when high abundance proteins, such as albumin, mask large sections of a gel. When masking occurs, other proteins of interest in these regions will not be detected. Cellular alterations affecting post-translational folding or glycosylation (glycoform) patterns cannot be resolved.

Second, there is loss of protein functional activity. Bio-activity cannot be measured because the proteins being resolved are denatured in 2DE.

Additionally, gel staining is imprecise and subject to interpretation. Also, costs tend to be prohibitively high. Processing of 2D gels is labor and time-intensive. Automation is not system-wide and requires expensive, highly specialized equipment.

High Performance Liquid Chromatography (HPLC), like 2DE, relies on the protein characteristics of charge and size. However, as it requires continuous gradient elution, many of these same constraints apply. In particular, it is difficult to implement HPLC in a high throughput fashion. Furthermore, at least in reverse phase mode, the proteins are denatured by use of organic solvents.

Mass spectrometry, also used to characterize protein molecules and their fragments, offers no possibility of assessing biological activity.

Certain recent proteomic platform technologies, such as biochips and microarrays, attempt to address throughput constraints with automated, high-content analysis. These devices are designed to select only for those pre-identified proteins selected to be applied to the device, thus providing a "closed" system. Along with traditional affinity chromatography, these platforms incorporate specialized, often laboriously designed and expensive bio-affinity ligands such as monoclonal antibodies, peptides, protein fragments, intact proteins, and particular low molecular weight ligands. They have limited scope for broad spectrum analyses, or wherever the content of the sample is largely unknown.

In view of the serious problems and deficiencies reviewed above, there remains a need for a proteomics analytical modality that is open-ended, rapid, convenient and suitable for implementation in a high throughput parallel assay system. There further is a need for proteomics compositions and methods that provide proteins resolved from a mixture in their native, biologically functional conformation. Additionally there is a need to characterize a protein, whether known or newly identified, according to its differential binding to specificity-determining substrates under varying ambient conditions, to provide a matrix or fingerprint of properties across the spectrum of interrogated environments. There still remains a need as well to identify particular protein species that are modulated positively or negatively in a sample such as a disease or pathological state with reference to a normal or nonpathological state. The present invention addresses these unfulfilled needs.

### Summary of Invention

In one aspect, the present invention provides a specificity-determining substrate that forms a complex with a protein molecule in a homogenous fashion. The specificity-determining substrate includes a specificity-determining ligand bound to a support, wherein optionally the substrate further includes a spacer bound between the ligand and the support. In this substrate the spatial separation between adjacent ligand groups is greater than a predetermined minimum distance.

In another aspect of the invention, a complex is provided that includes a specificity-determining substrate described in the preceding paragraph and a protein molecule.

In a further aspect of the invention, an array including a plurality of loci is provided, in which each locus includes a specificity-determining substrate described above. In advantageous embodiments of an array, the specificity-determining ligand at a first locus differs from a specificity-determining ligand at a second locus, and in other such embodiments the specificity-determining ligand at a first locus is identical to a specificity-determining ligand at a second locus.

In yet an additional aspect the present invention provides a method of resolving a first protein from a fluid including one or more species of native, biologically active protein molecules, wherein the first protein retains its native structure and its biological activity, the method including the steps of:

a) contacting the fluid with a specificity-determining substrate described above, thereby forming a complex including the first protein; and

b) separating the fluid so contacted from the complex;

thereby resolving the first protein from the fluid.

In important embodiments of the present method, the fluid includes a plurality of species of protein molecule, and the contacted and separated fluid includes a second protein.

In a further aspect, the present invention provides the method described immediately above that further includes, prior to performing step a) the fluid is pretreated by a method including the steps of:

a') contacting the fluid with a hydrogel including a water insoluble cross-linked polyhydroxy polycarboxylic acid having at least two strands each having a strand skeleton of the formula:

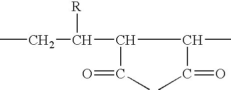

wherein R is H, OCH₃, or phenyl, and one carbonyl group of at least one maleoyl moiety thereof in each strand is covalently linked to a

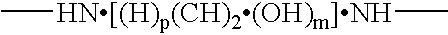

moiety to provide the presence therein of at least one cross linking moiety of the formula:

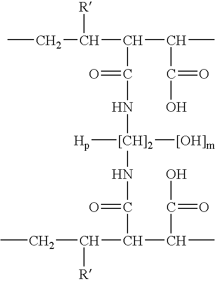

wherein R' is hydrogen or lower alkylene or lower alkoxy of 1-4 carbon atoms, or phenyl,

z is an integer of 1-4,

p is 0 or an integer up to z-1,

m is 1 or an integer up to z,

wherein the ratio of cross-links to poly (alkylene carbonic acid) strands is between about 1 and about 200 to 2,

to provide a hydrogel phase and a first supernatant; and

a") either

i) separating the hydrogel phase from the first supernatant and using the first supernatant as the fluid including one or more species of native, biologically active protein molecules, or

ii) separating the hydrogel phase from the first supernatant, then treating the hydrogel phase to release proteins adhering within it to provide a second supernatant including adhered proteins, and using the second supernatant as the fluid including one or more species of native, biologically active protein molecules.

In a still additional aspect, the present invention provides a method of purifying one or more first proteins from a fluid including one or more species of native, biologically active protein molecules, wherein the purified first protein retains its native structure and its biological activity, the method including the sequential steps of

a) contacting the fluid with a specificity-determining substrate as described herein, thereby forming a complex described including the one or more first proteins;

b) separating the contacted fluid from the complex; and

c) eluting the one or more first proteins from the specificity-determining substrate under conditions that retain the native structure and biological activity of the first protein;

thereby providing one or more purified native, biologically active first proteins. In an advantageous embodiment of the purifying method the fluid includes a plurality of species of protein molecule, and the contacted and separated fluid includes a second protein.

In still a further aspect the present invention provides a method of characterizing one or more proteins in a fluid including one or more species of protein molecule, the method including the sequential steps of

a) providing a plurality of containers, wherein each container has a characteristic specificity-determining substrate as described herein and a characteristic set of ambient fluid conditions, and wherein ambient fluid conditions are described by one or more variables chosen from the group consisting of the temperature, the ionic strength, the fluid composition, an amount of a chaotropic agent, an amount of a detergent, an amount of an organic cosolvent, and the pH, wherein each of said ligand and said ambient fluid conditions in a first container may be the same or different from said ligand and said ambient fluid conditions in a second container;

b) contacting the fluid with the plurality of containers, thereby promoting formation of a complex including the one or more proteins in a first container and inhibiting formation of a complex including the one or more proteins in a second container;

c) identifying the promotion of complex formation in the first container and the inhibition of complex formation in the second container; and

d) identifying the ligand and ambient fluid conditions in the first container and in the second container;

thereby characterizing the one or more proteins.

In yet a further aspect, the present invention provides a method of identifying one or more proteins in a sample fluid wherein the concentration of the one or more proteins in the sample fluid differs from the concentration of the one or more proteins in a reference fluid, the method including the sequential steps of

a) in a set of N containers, contacting the sample fluid with a specificity-determining substrate as described herein and an ambient fluid, wherein the ambient fluid has conditions described by one or more variables chosen from the group consisting of the temperature, the ionic strength, the fluid composition, an amount of a chaotropic agent, an amount of a detergent, an amount of an organic cosolvent, and the pH, wherein each container is characterized by a particular specificity-determining substrate and a particular fluid condition, the particular substrate and particular fluid conditions potentially promoting formation of a complex including the one or more proteins;

b) determining the amount and/or species of protein molecules complexed with the substrate in each container of the sample fluid set;

c) comparing the amount and/or species of protein molecules complexed with the substrate in each container of the sample set with the amount and/or species of protein molecules complexed with the substrate obtained by contacting a reference fluid with a specificity-determining substrate as described herein and an ambient fluid in a reference set of containers identical to the set used for the sample fluid, wherein the i-th container in the sample set and the i-th container in the reference set have the identical specificity-determining substrate and the identical ambient fluid conditions (1 ≤ i ≤N);

d) identifying an n-th container in the sample fluid set whose amount of one or more proteins complexed with the substrate differs from the amount of the one or more proteins complexed with the substrate in the n-th container in the reference fluid set (1 ≤ n ≤N); and

e) identifying the one or more proteins as the species whose amounts complexed with the substrate differ between the n-th sample container and the n-th reference container.

In all the various substrates, complexes, arrays and methods provided above, in important embodiments the specificity-determining ligand is chosen from the group consisting of a monosaccharide, a disaccharide, a trisaccharide, an oligosaccharide,

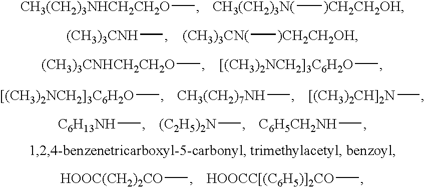

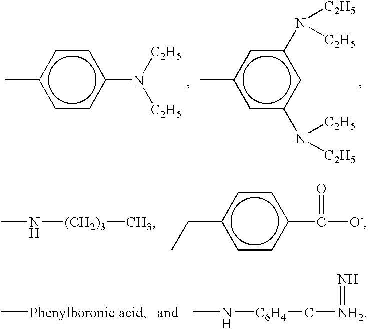

In additional significant embodiments the specificity-determining ligand includes

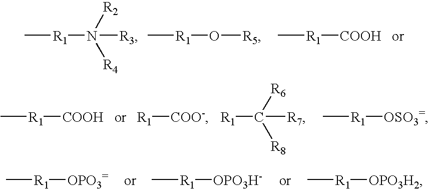

wherein R₁ is chosen from the group consisting of a normal or branched aliphatic moiety, a cycloalkyl moiety, an aromatic moiety, an aralkyl moiety, an aliphatic-aliphatic ether, an aliphatic-aromatic ether, an aliphatic moiety having a secondary or tertiary alcohol, a phenolic moiety, an aliphatic moiety having a secondary or tertiary amine, an aniline moiety, an aliphatic carboxyl, sulfate, sulfonate, or phosphate ester, an aromatic carboxyl, sulfate, sulfonate or phosphate ester, and an aromatic heterocycle;and

wherein R₂, R₃, R₄, R₅, R₆, R₇, and R₈, are independently chosen from the group consisting of hydrogen, a normal or branched aliphatic radical, a cycloalkyl radical, an aromatic radical, an aralkyl radical, an aliphatic-aliphatic ether, an aliphatic-aromatic ether, an aliphatic radical having a primary, secondary or tertiary alcohol, a phenolic radical, an aliphatic radical having a primary, secondary or tertiary amine, an aniline radical, an aliphatic carboxylate, sulfate, sulfonate, or phosphate group, an aromatic carboxylate, sulfate, sulfonate or phosphate group, and an aromatic heterocycle.

In still additional advantageous embodiments a spacer employed in the substrate is

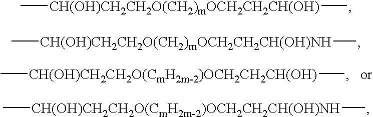

where m is an integer between 2 and 10.

In further significant embodiments the support is chosen from the group consisting of a glass surface, a silica surface, a ceramic surface, a plastic surface, a resin particle, a bead, a gel, a polyelectrolyte, and a hydrogel. In certain advantageous embodiments the support is other than a surface and wherein the solids content of the support when equilibrated with an ambient fluid is less than a predetermined maximum content; for example, the solids content of the support may be less than about 8% w/v.

In particular significant embodiments of the invention the support includes a polysaccharide; and in certain advantageous embodiments the support includes chitosan.

### Brief Description of Drawings

Fig. 1. SDS-PAGE profiles of sheep plasma prior to and after treatment with crosslinked polyhydroxy polycarboxylic acid (CPPA).

Lane 1: Untreated plasma stained for protein with Coomassie Blue;

Lane 2: CPPA treated plasma stained for protein with Coomassie Blue;

Lane 3: Untreated plasma stained for glycoproteins using Periodate Schiff reagent;

Lane 4: CPPA treated plasma stained for glycoproteins using Periodate Schiff Reagent.

Fig. 2. Production Capacity of Chitosan Hydrogel at various pH values and buffer concentrations. Test conditions: Column Size: 1.5 cm Dia. x 17 cm Ht. (30 mLs packed bed volume); Flow Rate: 5 mL/min; Residence Time: 6 minutes; Protein Concentration: 2 mg/mL; Process Yield: >90%; Buffers: Potassium Phosphate.

Fig. 3. Purification of Serum Polyclonal Antibodies with Chitosan DEA.

Chitosan DEA chromatography and SDS-PAGE of hyper-immune goat serum at 6 minute load cycle Residence Time. Test Conditions: Load: Dialyzed Serum, Flow Through = Flow Through + Wash; Equilibration and Wash Buffer: 10 mM Potassium Phosphate (monobasic)/NaOH, pH 6.0; Elution Buffer: 10 mM Potassium Phosphate (monobasic)/NaOH, pH 6.0, 0.5M NaCl.

Fig. 4. Purification of Serum Polyclonal Antibodies with Chitosan-COO. Panel A. Production capacity of Bovine Serum Albumin and IgG. Panel B. Chitosan-COO chromatogram illustrating elution of immunoglobulin. Panel C. Chromatogram and PAGE of purified monoclonal antibody from cell culture grown in media containing 10% fetal calf serum using a moderately hydrophobic, anionic ligand.

Fig. 5. Relative scores of elution of bovine serum albumin, glycated bovine serum albumin and rabbit serum albumin from chitosan-COO.

Fig. 6. SDS-PAGE electrophoretograms for four chitosan supports bearing four different specificity-determining ligands. The left and center panels were developed with Coomassie Blue. The right panel was developed with periodate Schiff colorimetric stain.

Fig. 7. SDS-PAGE electrophoretograms of a protein mixture for chitosan supports derivatized with various ligands. The designation "Pro" represents the untreated mixture of transferrin, albumin, and ovalbumin. Panel A. Characterization of Ligands 1,2,4,5,and 6. Panel B. Characterization of Ligands A, B, C, D, E, F, I, J, K, 1,2,4,5,and 6.

Fig. 8. SDS-PAGE electrophoretograms of sheep plasma after enrichment of the glycoprotein fraction for chitosan supports derivatized with various ligands. The designations "IgG" "Pls" and "GP" respectively, represent an IgG reference, Plasma Control (1:10 dilution), and glycoproteins enriched after acidic polyelectrolyte hydrogel treatment. Left panels, Coomassie Stain, right panels, Periodate Schiff Stain. Panel A. Characterization of Ligands A, B, C, D, E, F, and I. Panel B. Characterization of Ligands J, K, 1,2,4,5,and 6.

Fig. 9. SDS-PAGE of Plasma dosed with horseradish peroxidase, and peroxidase activity recovered, using four ligands bound to chitosan specificity-determining substrate.

Fig. 10. Glyco-Proteomic System Schematic Diagram.

### Detailed Description

The present invention provides a variety of related proteomics analytical modalities that are open-ended, rapid, convenient and suitable for implementation in a high throughput parallel assay system. Specificity-determining compositions and methods are disclosed for use in proteomics. These compositions and methods provide a protein resolved from other protein species contained in a sample fluid, in its native, biologically functional conformation. Additionally this invention provides compositions and methods for characterizing a protein, whether known or newly identified, according to its differential binding properties in a related set of ambient conditions, to provide a matrix or fingerprint of its properties across the spectrum of environments investigated. The present invention additionally discloses compositions and modalities for identifying particular protein species whose amount is modulated positively or negatively (including whether it is presence or absent) in a sample such as a disease or pathological state, in comparison with a reference to a normal or nonpathological state.

Most protein separation methods take advantage of the differences in the number and distribution of charged, polar and apolar amino acid residues arrayed along the primary sequence and displayed on the surface structure of a native protein. Many commercially available supports for protein analysis in chromatographic formats utilize small, dense, highly crosslinked polysaccharides or synthetic polymers containing micropores. Although these beads or matrices have a high surface area density that provides a large number of sites for ligand attachment, the bead surface also has the property that it promotes undesirable protein/protein aggregation within the aqueous microenvironment of the bead. Furthermore, closely juxtaposed ligand-protein complexes on the bead surface interact strongly with each other; a condition that makes the subsequent recovery of the desired protein difficult even under denaturing elution conditions. In addition, because of the dense structure there is the possibility of nonspecific interaction of a protein solute with the support substance, as well as for diffusion into dead-end pores. The present inventors term this complex set of phenomena "heterogeneous binding" of a protein solute and a solid support. Without wishing to be bound by theory, they believe that such effects contribute to binding of a given protein on such supports with excessively variable affinities, leading to a requirement for a broad range of conditions for eluting the protein from the support.

The inventors, while not wishing to be bound by theory or hypothesis, have designed very low density specificity-determining substrates with an open architecture. This is intended to overcome perceived problems such as site-site interactions between bound protein solutes, and nonspecific binding. This objective is accomplished in the present invention by having a solids content of the support, when equilibrated with an ambient fluid, less than a predetermined maximum content. In certain embodiments, including those in which the support is not a solid surface, the predetermined maximum solids content may be less than about 12% w/v, or less than about 10% w/v, or less than about 8% w/v, or less than about 7% w/v, or less than about 6% w/v, or less than about 5% w/v, or less than about 4% w/v, or less than about 3% w/v, or less than about 2% w/v, or less than about 1% w/v.

Additionally the inventors, while not wishing to be bound by theory or hypothesis, have spaced protein-binding ligands on a specificity-determining substrate to minimize non-specific interaction effects. This may be achieved by having a spatial separation between adjacent ligand groups be greater than a predetermined minimum distance. In various embodiments the predetermined minimum distance separating adjacent ligand groups may be greater than about 4Å, or greater than about 5 Å, or greater than about 6 Å, or greater than about 8 Å, or greater than about 10 Å, or greater than about 12 Å, or greater than about 15 Å, or greater than about 20 Å, or greater than about 25 Å, or even greater. As a consequence of the extended spacing between ligand groups on the support, the binding capacity expressed as moles protein bound per gm dry weight of the specificity-determining support or as moles protein bound per mL of the fluid-equilibrated specificity-determining support, generally speaking, is intentionally kept relatively low. The properties characterizing the support structures disclosed here, that are believed to improve resolution, are collectively referred to as fostering "homogenous" binding herein.

As used herein including the claims, the article "a" when used to describe an item or object, refers both to the singular and plural forms of the item or object. As an example the phrase "a protein" may designate either one protein or a plurality of proteins.

The present invention discloses a specificity-determining substrate that forms a complex with a protein molecule in a homogenous fashion. The specificity-determining substrate includes in its structure a specificity-determining ligand bound to a support, and optionally the substrate further includes a spacer bound between the ligand and the support. As indicated above, the specificity-determining substrate is characterized by a spatial separation between adjacent ligand groups that is greater than a predetermined minimum distance, and for supports that are not a solid surface by having a solids content of the support when equilibrated with an ambient fluid being less than a predetermined maximum content.

A spacer may be included in the specificity-determining substrate, interposed between the support and the specificity-determining ligand. A spacer contributes to accomplishing the objective of promoting homogeneous binding of a protein to a ligand. Spacers are widely known and practiced in fields related to those of the present invention, including diagnostic biochemistry, analytical biochemistry, biochemical separations, protein chemistry, and related fields. In certain embodiments of the present invention, a spacer is

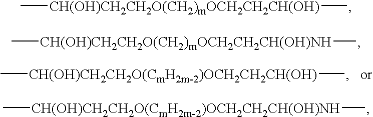

where m is an integer between 2 and 10.

The spatially separated specificity-determining ligand used in a given substrate is chosen to contribute to establishing the specificity of the substrate for the proteins it binds. In order to accomplish this specificity, the ligand may be any moiety that confers this specificity. By varying physical and chemical characteristics of a specificity-determining ligand, a given ligand binds a variety of different proteins. Such features include, by way of nonlimiting example, ionic groups, hydrogen bonding donors and acceptors, nonpolar moieties that in water contribute to hydrophobic interactions, and aromatic groups. A reactive precursor of a ligand includes a reactive group for coupling to the support, or to the terminus of a spacer moiety, that is chosen to target a functional group presented by the support or spacer. Workers of skill in fields such as synthetic organic chemistry, pharmaceutical chemistry, polymer chemistry, protein chemistry, and other fields related to the field of the present invention are adept at designing and/or identifying suitable ligands for coupling to a support of the invention. In certain embodiments, the specificity-determining ligand is chosen from among a monosaccharide, a disaccharide, a trisaccharide, an oligosaccharide,

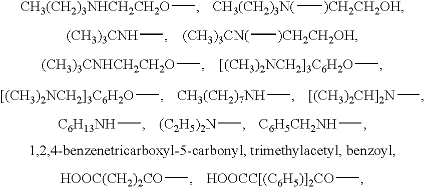

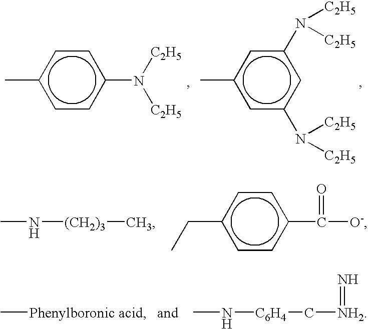

In additional embodiments, the specificity-determining ligand includes

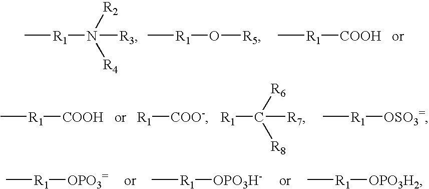

wherein R₁is chosen from the group consisting of a normal or branched aliphatic moiety, a cycloalkyl moiety, an aromatic moiety, an aralkyl moiety, an aliphatic-aliphatic ether, an aliphatic-aromatic ether, an aliphatic moiety having a secondary or tertiary alcohol, a phenolic moiety, an aliphatic moiety having a secondary or tertiary amine, an aniline moiety, an aliphatic carboxyl, sulfate, sulfonate, or phosphate ester, an aromatic carboxyl, sulfate, sulfonate or phosphate ester, and an aliphatic or aromatic heterocycle;

and wherein R₂, R₃, R₄, R₅, R₆, R₇, and R₈, are independently chosen from the group consisting of hydrogen, a normal or branched aliphatic radical, a cycloalkyl radical, an aromatic radical, an aralkyl radical, an aliphatic-aliphatic ether, an aliphatic-aromatic ether, an aliphatic radical having a primary, secondary or tertiary alcohol, a phenolic radical, an aliphatic radical having a primary, secondary or tertiary amine, an aniline radical, an aliphatic carboxylate, sulfate, sulfonate, or phosphate group, an aromatic carboxylate, sulfate, sulfonate or phosphate group, and an aliphatic or aromatic heterocycle.

In certain embodiments of the present invention the specificity-determining substrate is based on a substrate that includes chitosan. Chitosan is a polysaccharide with pendant amine groups. Without wishing to be bound by theory, the present inventors believe that use of a spacer interposed between the chitosan support and a specificity-determining ligand effectively places the ligand out of the range of ionic interactions with the chitosan amine groups. The specificity of location of active sites in combination with the location of these active sites away from the chitosan matrix improves the specificity of binding and desorption. In the present invention when the support is crosslinked chitosan with a spacer, the ligand is other than a diethylamino or diethylaminoethyl group.

The Examples provided below demonstrate generally that upon resolving a protein from a fluid using a specificity-determining substrate of the present invention, described above, a complex of the present invention is provided that includes the substrate and the protein molecule in isolation. Specifically, after a sample fluid has been treated with a specificity-determining substrate and the treated fluid is subsequently withdrawn, the complex remains behind. In important embodiments of such complexes, the bound protein is in such a conformational state that upon eluting the protein from the substrate, the protein molecule is not denatured or inactivated. Rather, conformational integrity and biological activity are retained. Without wishing to be bound by theory, a complex of the present invention is believed to form as a result of the binding of the specificity-determining ligand to a protein. A protein that binds is though to have a surface whose physical and chemical characteristics complement those of the ligand sufficiently to establish an affinity for the protein. There is no limitation or restriction, in general, on the range or variety of proteins that may bind to various ligands that may be employed in the present invention. This broad scope of potential binding interactions contributes significantly to the use of the present compositions and methods in proteomics investigations.

The choices of specificity-determining ligand, spacer, and support are as have already been described for the present invention in the paragraphs above with respect to the substrate alone. That complete discussion is incorporated here by reference.

The invention further provides a mixture that includes a fluid that contains at least one species of protein molecule, and also contains a specificity-determining substrate. In the disclosed mixture, the protein molecule and the substrate form a complex, as described in the preceding paragraphs. In certain embodiments the fluid is an aqueous preparation containing at least one protein molecule, and in many embodiments the fluid contains many species of protein molecule. In such embodiments one or more of the protein species do not form a complex with the substrate described above. These embodiments relate to the use of the substrate compositions of the invention, their complexes, and their mixtures, in processes that resolve or purify a particular protein species from a sample fluid that is composed of many protein species.

In important embodiments of this invention, the compositions and methods relate to high throughput modalities that are commonly used in proteomics research. Accordingly, the invention additionally provides several mixtures, with each mixture being as described above. In high throughput systems, each mixture is placed into a separate container such as a well of a microtiter plate, or is created at a locus in a printed or deposited array. Commonly, each mixture in an array, or at least at several loci of the array, includes the same specificity-determining ligand. For those loci with the same ligand, fluid conditions that may be described by specifying one or more variables chosen from among the temperature, the ionic strength, the fluid composition, an amount of a chaotropic agent, an amount of a detergent, an amount of an organic cosolvent, the pH, and similar ambient variables, may be varied, such that the variables describing a first mixture differ from the variables describing a second mixture. In complementary embodiments, several loci of the array may have differing specificity-determining ligands but the same fluid conditions. In these ways a mathematical matrix describing the binding properties characterizing a particular protein-ligand complex may be obtained incorporating information on the ligand, fluid conditions and binding properties of each locus; alternatively, this matrix may be thought of as providing a "fingerprint" of the binding interaction.

The invention further provides an array that includes several loci each of which includes a specificity-determining substrate described above. In common embodiments of an array, the identity of the specificity-determining ligand at one locus differs from the identity of a specificity-determining ligand at another locus in the array. In other common embodiments of an array, the specificity-determining ligand at one locus is identical to the specificity-determining ligand at one or more other loci. The details of the construction of a specificity-determining substrate used at each locus of an array, relating to choice of ligand, choice of spacer and choice of support, are as have already been described above, as have the attributes of the complexes, and of the mixtures, that may be found at each locus of the array.

The present invention discloses various ways in which the above compositions and arrays may be used. First, the invention provides a method of resolving a protein from a fluid that contains one or more species of native, biologically active protein molecules. Upon release of the protein from binding to form a complex the resolved protein retains its native structure and its biological activity. In implementing this method the steps employed include adding the fluid to a preparation of a specificity-determining substrate of the invention and constituted as described above. In this way a complex of the invention as described above, that includes the protein, is formed. The fluid and the substrate are allowed to interact under conditions that promote complex formation, and for a time sufficient for complex formation to be completed. By way of nonlimiting example this may range from 5 min or less to 60 min or more. The mixture of the fluid and substrate is typically stirred, agitated or similarly manipulated to ensure thorough contact between these components. Subsequently the treated fluid is separated from the complex. The fluid may be separated by sedimenting a substrate that is a bead or particle, by decanting the fluid from the substrate, by aspiration,or similar suitable means. In this way the protein is resolved from the fluid.

In many embodiments of the method of resolving a protein, the fluid is a sample that contains many species of protein molecule, only some of which bind to the substrate. In such cases, the contacted and separated fluid contains the remaining unbound protein species.

In many significant embodiments of the method of resolving a protein, the fluid containing the one or more protein species is first treated in a way that preferentially enriches certain classes of proteins, such as glycoproteins, in an initial supernatant, and preferentially enriches other classes of proteins, such as nonglycosylated protein species immobilized in a cross-linked polyhydroxy polycarboxylic acid. The CPPA and this preferential enrichment process is disclosed in U. S. Pats. 5,294,681, 5,453,493, 5,534,597, and 5,976,382, each of which is incorporated herein by reference in its entirety. Thus, by way of nonlimiting example, one implementation of this preliminary enrichment procedure includes the following steps performed before the steps of the resolving method described above:

a') contacting the fluid with a hydrogel whose structure includes a water insoluble cross-linked polyhydroxy polycarboxylic acid having at least two strands, each strand having a strand skeleton of the formula:

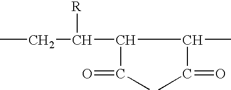

wherein R is H, OCH₃, or phenyl, and one carbonyl group of at least one maleoyl moiety thereof in each strand is covalently linked to a

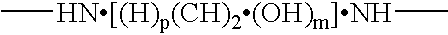

moiety to provide the presence therein of at least one moiety that crosslinks two strands according to the structural formula:

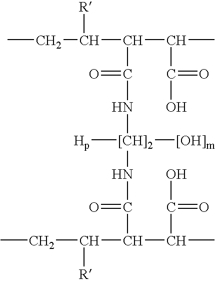

wherein R' is hydrogen or lower alkylene or lower alkoxy of 1-4 carbon atoms, or phenyl,

z is an integer of 1-4,

p is 0 or an integer up to z-1,

m is 1 or an integer up to z,

and wherein the ratio of cross-links to poly (alkylene carbonic acid) strands is between about 1 and about 200 to 2,

to provide a hydrogel phase and a first supernatant; and

a") either

i) separating the hydrogel phase from the first supernatant and using the first supernatant as the fluid to be used in the method of resolving described above, or

ii) separating the hydrogel phase from the first supernatant, then treating the hydrogel phase to release proteins adhering within it to provide a second supernatant comprising adhered proteins, and using the second supernatant to be used in the method of resolving described above. Procedures that are employed to release proteins adhering within the hydrogel are disclosed in U. S. Pats. 5,294,681, 5,453,493, 5,534,597, and 5,976,382.

In other embodiments additional types of interstrand crosslinks in the CPPA may be introduced as well (U. S. Pats. 5,453,493 and 5,976,382).

The present invention also provides a method of purifying a protein from a fluid that contains one or more species of native, biologically active protein molecule. In many embodiments of this method the fluid contains several species of protein molecule; after treatment the resulting fluid contains many of the protein species not purified from the fluid. Additionally, the purified protein retains its native structure and its biological activity. As used herein, "purifying" and related terms indicate that a substance, originally found in a fluid or in a sample that contains at least one other protein, or at least one other nonprotein substance, is in a fluid or other form from which a significant proportion of the other proteins or other nonprotein substances have been separated.

This method includes the sequential steps of

a) contacting the fluid with a specificity-determining substrate of the present invention, thereby forming a complex provided by the present invention that includes the protein being purified;

b) separating the contacted fluid from the complex; and

c) eluting the protein from the specificity-determining substrate under conditions that retain the native structure and biological activity of the protein,

thereby providing a purified native, biologically active protein.

The method of purifying a native, biologically active protein may be preceded by a pretreatment that provides a fluid that is enriched in either glycosylated proteins or nonglycosylated proteins as described above and set forth in detail in U. S. Pats. 5,294,681, 5,453,493, 5,534,597, and 5,976,382, incorporated herein by reference.

A purified protein may be further characterized by methods and techniques well known to workers of skill in fields related to the present invention, including, by way of nonlimiting example, protein chemistry, protein structure, cell biology, pharmacology, oncology, and so forth. For example, a protein may be further studied to identify or assay its molecular weight, its amino acid sequence, the nature and position of conjugated modifying moieties, its chromatographic characteristics, and its biological function or activity. The latter may include binding activities, enzymatic activities, receptor activities, signal transduction activities, and so forth.

The present invention additionally provides a method of characterizing one or more proteins in a fluid that contains one or more species of protein molecule. The method includes the sequential steps of

a) First, several containers are provided, each container having a characteristic specificity-determining substrate described in the present invention, above, and a characteristic set of ambient fluid conditions described by one or more variables chosen from among the temperature, the ionic strength, the fluid composition, an amount of a chaotropic agent, an amount of a detergent, an amount of an organic cosolvent, and the pH. In a particular container each of the ligand and the ambient fluid conditions may be the same or different from the ligand and the ambient fluid conditions that characterize a second container.

b) Second, the fluid is contacted with each of the containers, thereby promoting formation of a complex including the one or more proteins in a first container and inhibiting formation of a complex including the one or more proteins in a second container.

c) Third, the promotion of complex formation in the first container and the inhibition of complex formation in the second container is identified.

d) Fourth, the ligand and ambient fluid conditions in the first container and in the second container are identified; thereby characterizing the one or more proteins.

Thus, in various embodiments of the method of characterizing a protein, the method may involve a set of containers having differing specificity-determining substrates, while the ambient fluid conditions are the same for all the containers; or the same substrates and differing ambient fluid conditions; or different substrates and different ambient fluid conditions. In these embodiments the characterization of the protein includes the identities of the specificity-determining ligands employed in the specificity-determining substrate, as well as the ambient fluid conditions, that promote and that inhibit the formation of the complex.

A broad range of techniques and procedures are available to identify whether complex formation has ensued. These are all well known to workers of skill in fields related to the present invention including biochemistry, protein chemistry, protein structure, protein physical chemistry, enzymology, proteomics, cell biology, pharmacology, immunology, oncology, and so forth. Nonlimiting examples of such techniques may be chosen from physical, chemical and biochemical procedures. Physical techniques include, for example, spectroscopy of all kinds including optical, infrared, electron spin resonance, magnetic resonance, and so forth; scattering methods such as laser light scattering; total internal reflectance spectroscopy using either absorption or fluorescence, surface plasmon resonance, and so forth. Chemical methods include, for example, staining or colorimetric methods. Biochemical methods include, by way of example, binding and enzymatic assays, immunochemical assays, receptor and signal transduction assays, and the like. Any technique or method that equivalently contributes to determining that complex formation is promoted or inhibited is included within the scope of the present method of characterizing a protein.

The method of characterizing a protein in a fluid is well suited for proteomics investigations. It is readily implemented in a high throughput format such as a microtiter plate or an array. The specificity-determining substrate may be provided in the form of beads or particles, or in the form of a surface to which the specificity-determining substrate is bound. The various techniques for identifying complex formation lend themselves to these various formats advantageously.

The method of characterizing a protein in a fluid may be preceded by a pretreatment that provides a fluid that is enriched in a particular class of protein, such as glycosylated proteins or nonglycosylated proteins, or phosphoproteins vs nonphosphorylated proteins, as described above (see U. S. Pats. 5,294,681, 5,453,493, 5,534,597, and 5,976,382, incorporated herein by reference).

The present invention further discloses a method of determining that the concentration or prevalence of a particular protein or proteins, present in a sample fluid, differs from its concentration or prevalence in an appropriately chosen reference fluid. This method includes the sequential steps:

a) First, in a set of several containers, the sample fluid is brought into contact with a specificity-determining substrate of the invention as described above, and with an ambient fluid; the ambient fluid has conditions described by one or more variables chosen from among the temperature, the ionic strength, the fluid composition, an amount of a chaotropic agent, an amount of a detergent, an amount of an organic cosolvent, the pH, and similar environmental variables. In the set, each container is characterized by a particular specificity-determining substrate and a particular fluid condition, such that these attributes potentially promote formation of a complex of the invention described above that includes the protein.

b) Second, the amount and/or species of protein molecules complexed with the substrate in each container of the sample fluid set is determined.

c) Third, either earlier or at the same time, a reference fluid derived or obtained from a suitable reference sample has been or is brought into contact with a specificity-determining substrate of the invention as described above, and with an ambient fluid, in a set of containers (the reference set) identical to the set used for the sample fluid. Thus, the i-th container in the sample set and the i-th container in the reference set have the identical specificity-determining substrate and the identical ambient fluid conditions. The amount and/or species of protein molecules complexed with the substrate in each container of the sample set is compared with the amount and/or species of protein molecules complexed with the substrate in the corresponding container of the reference set.

d) Fourth, an n-th container in the sample fluid set whose amount of the protein complexed with the substrate differs from the amount of the same protein complexed with the substrate in the n-th container in the reference fluid set is identified.

e) Fifth, the protein is identified as the species whose amount complexed with the substrate differs between the n-th sample container and the n-th reference container.

This method of determining differences in the concentration or prevalence of a particular protein is of great significance in the proteomics field. Since it is readily suited to be performed in a high throughput format such as a microtiter plate or an array, a differentially occurring protein may be readily identified. This method is rapid, inexpensive and in many embodiments may be carried out with apparatus that is also inexpensive. Furthermore, an important advantage of the method is that it is open-ended, rather than operating in a closed system. In contrast, a proteomics array of proteins or protein fragments is considered a closed system, in the sense that detection is limited to the proteins or fragments that may be introduced ahead of time in preparing the array. The present method is open-ended in that no limitation exists on the ability to identify a protein that occurs differentially in a sample vs a reference; potentially any such protein is susceptible of identification by the present method. It therefore offers a highly advantageous means for identifying potential novel or uncharacterized targets for treatment of a disease or pathology.

The method of determining differences in the concentration or prevalence of a particular protein may be preceded by a pretreatment that provides a fluid that is enriched in either glycosylated proteins or nonglycosylated proteins, as described above and set forth in detail in U. S. Pats. 5,294,681, 5,453,493, 5,534,597, and 5,976,382, incorporated herein by reference.

A broad range of techniques and procedures are available to identify whether complex formation has ensued, and whether determining differences in the concentration or prevalence of a particular protein. These are all well known to workers of skill in fields related to the present invention including biochemistry, protein chemistry, protein structure, protein physical chemistry, enzymology, proteomics, cell biology, pharmacology, immunology, oncology, and so forth, and have been discussed in detail above.

EXAMPLES

The following Examples are offered to illustrate particular embodiments of the present invention. The Examples in no way limit the scope of the invention as set forth in the instant claims.

Reference Example 1.

A 2.5 w/v% aqueous suspension of a water insoluble cross-linked polyhydroxy polycarboxylic acid hydrogel which was prepared by reacting methoxy ethylene maleic anhydride with diamino hydroxy propane according to the methods previously described (US Patent Nos. 5,294,681, 5,453,493, and 5,534,597; herein termed "crosslinked polyhydroxy polycarboxylic acid (CPPA)" '). The suspension was centrifuged at 2000 x g for 10 minutes, the pellet was recovered and its pH recorded. In another experiment a 30 mg/mL solution of bovine serum albumin (BSA) in water was treated with an equal volume of CPPA suspension. The contents were mixed by repeated inversions for 10 minutes and then centrifuged. The clear supernatant and pellet (CPPA - BSA aggregate) were recovered and their respective pH values determined. The amount of BSA removed from solution after CPPA treatment was also recorded. The results of these determinations are given in Table 1.

TABLE 1

| Sample | pH | % Protein removed |
|---|---|---|
| CPPA supernatant | 4.5 | N/A |
| CPPA pellet | 3.1 | N/A |
| BSA solution 30 mg/ml in water | 5.4 | N/A |
| Supernatant of BSA solution after CPPA | 4.1 | 99.6 |
| treatment | | |
| CPPA BSA pellet | 4.2 | N/A |

N/A: not applicable or not determined.

Reference Example 2. Glycoprotein Enrichment Efficiency

The elastomeric polyelectrolyte CPPA had previously been evaluated for its ability to bind and aggregate diverse proteins contained in phosphate buffered saline pH 7.3 (US Patent Nos. 5,294,681, 5,453,493, and 5,534,597). It was found that nonglycosylated proteins such as human serum albumin and hemoglobin or marginally glycosylated proteins (e.g., immunoglobulins G) were removed at levels of 90 percent or above by CPPA. By contrast highly glycosylated proteins such as alpha 1-acid glycoproteins, fetuin and horseradish peroxidase did not bind to CPPA to any appreciable extent.

CPPA has been shown to have a high affinity for nonglycosylated proteins and a very low affinity for glycosylated proteins. Table 2 demonstrates this utility for pure isolates.

TABLE 2

| | | Protein | |
|---|---|---|---|
| | Initial protein | concentration | |
| | concentration | (mg/mL) after | % Protein |
| | (mg/mL) | 1:1 v/v CPPA | removed |
| Protein Isolates < 15% | | | |
| carbohydrate | | | |
| Human Serum Albumin | 30.0 | 0.15 | >99.0 |
| Human Immunoglobulin | 10.0 | 0.05 | 90.0 |
| Cytochrome C | 10.0 | 0.03 | >99.0 |
| Hemoglobin | 30.0 | 0.03 | >99.0 |
| Protein Isolates > 20% | | | |
| carbohydrate | | | |
| Alpha 1 acid | 10.0 | 8.0 | 20.0 |
| glycoproteins | | | |
| Fetuin | 10.0 | 6.7 | 33.0 |
| Heman Chorionic | 10.0 | 8.3 | 17.0 |
| Gonadotropin (HCG) | | | |
| Horse radish peroxidase | 10.0 | 9.1 | 9.0 |
| bovine submax. mucin | 10.0 | 8.6 | 14.0 |

From these results it is seen that CPPA effectively sequesters nonglycosylated or weakly glycosylated proteins, whereas other known glycoproteins remain primarily in the supernatant phase.

Reference Example 3. Glycoproteins Added to Serum.

This Example assesses the ability of CPPA selectively to aggregate nonglycosylated proteins when added to a biological extract containing a variety of proteins, both nonglycosylated and glycosylated.

Four separate samples of serum (rabbit) were dosed with the glycoproteins (final serum concentrations in parenthesis) alpha-1 acid glycoproteins (40ug/mL), apo-transferrin (60ug/mL), fetuin (40ug/mL), and immunoglobulins G (80ug/mL).

An equal volume of CPPA was added to each of the dosed serum samples along with a control (undosed) serum sample treated with CPPA, and an untreated serum control. The tubes were mixed by repeated inversions for 15 minutes and then centrifuged at 10,000 x g for 10 minutes.

The supernatants were recovered and analyzed for protein using BCA Protein Assay kit and for glycoproteins using the Glycoprotein Carbohydrate Estimation kit (Pierce Chemicals, Rockford, IL).

It was found that total glycoproteins recovered from non-dosed sera treated with an equal volume of CPPA ranged from 70-76%. Total protein removed from serum treated with an equal volume of CPPA ranged from 75 to 81%.

The glycoproteins recoveries from serum dosed with the different glycoproteins are reported in Table 3.

TABLE 3

| Glycoprotein | % Recovery |
|---|---|
| Alpha 1 acid glycoproteins | 70-75 |
| Apo - transferin | 67-71 |
| Fetuin | 70-76 |
| Immunoglobulin G | 53-61 |

Reference Example 4. Glycoproteins Added to Plasma

Equal volumes of CPPA were added to equal volumes of the different plasma specimens. The tubes were mixed by repeated inversions for 15 minutes and then centrifuged at 10, 000 x g for 10 minutes. The supernatants were recovered and analyzed for protein and glycoproteins as previously described.

Sodium dodecyl sulfate (SDS) gel electrophoresis was performed on the sheep plasma before and after CPPA treatment. The samples were diluted 1:5 with electrophoresis buffer which contained 25mM Tris, 192mM glycine and 0.1% (w/v) SDS pH 8.3. Electrophoresis was conducted at 125 V for 90 minutes. The gels were subsequently stained for protein using Coomassie Blue and for glycoproteins using the Periodate Schiff from Pierce Chemicals.

The percent removal of total protein and the percent recovery of glycoproteins from plasma treated with CPPA are shown in Table 4.

TABLE 4

| Plasma Sample | % Total Protein Removed | % Glycoprotein Recovered |
|---|---|---|
| Bovine | 78.04 | 79.57 |
| Mouse | 87.40 | 96.53 |
| Rabbit | 87.42 | 87.21 |
| Goat | 78.73 | 74.61 |
| Sheep | 78.73 | 88.93 |

The total protein removed from the plasmas obtained from the different animals ranged from 78% to over 87%. The glycoproteins recoveries ranged from 79% to over 81%.

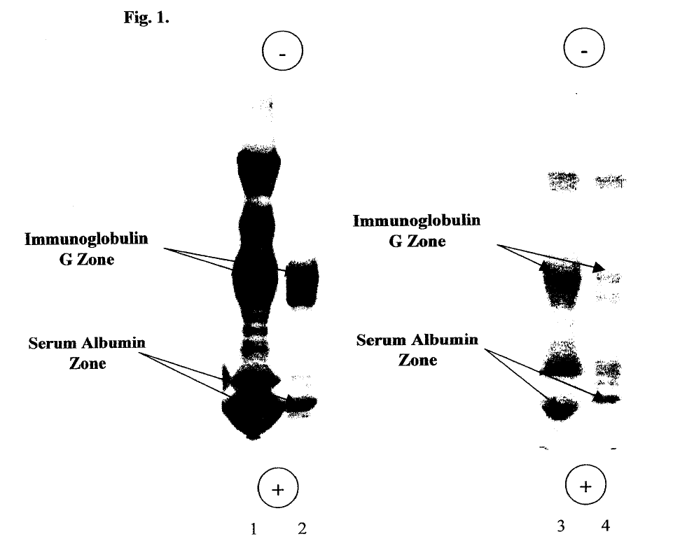

The electrophoretic patterns obtained for plasma before and after treatment with CPPA are shown in Fig. 1.

Comparing lanes 1 and 2, which are stained with Coomassie Blue, the CPPA removes most of the albumin, a nonglycosylated protein, and much of the immunoglobulin, a weakly glycosylated protein.

Comparing lanes 3 and 4 , which are stained by periodate Schiff base staining, shows similar glycoprotein profiles. The CPPA treatment has not removed the glycoproteins. The bands in the immunoglobulins G region are a little weaker for lane 2 indicating some losses of this marginally glycosylated protein as a result of CPPA treatment.

These electrophoretic profiles confirm the removal of copious quantities of albumin which is the highest abundance protein in serum, by the CPPA, along with partial removal of the immunoglobulins globulin fraction which contains 5-10% carbohydrate. The enrichment process clearly produces differentiation of several bands that are masked by the presence of albumin. The treatment also retains much of the glycoprotein species in the supernatant.

Reference Example 5. Glycoprotein Recovery And Protein Removal From The Membrane Fraction Of Animal Tissue.

In this Example, an attempt was made to recover detergent-solubilized glycoproteins from tissue extracts and to aggregate detergent-solubilized nonglycosylated proteins by CPPA treatment.

1). Ten grams of frozen rat livers were cut into small pieces with a scalpel blade and then transferred to a Cuisnart™ chopper/grinder Model Number MM-2 containing 100 mL of 10 mM Tris buffer pH 8.0. This preparation was homogenized using three intermittent 10 second pulses. This preparation was then centrifuged at 3000 x g for 10 minutes and both the supernatant and pellet recovered.

2). Equal volumes of CPPA and supernatant were combined in a tube. The tube was mixed by repeated inversion for 15 minutes and then centrifuged at 10,000 x g for 10 minutes. The supernatants were recovered and analyzed for protein and glycoproteins.

3) The particulate fraction from rat liver homogenate suspended in 40 milliliters of 1.0% Tween 20 in 10 mM Tris buffer pH 8.0 was mixed by repeated inversions for 30 minutes and then centrifuged at 3,000 x g for 30 minutes. The supernatant was recovered and treated with an equal volume of CPPA previously equilibrated with 1% Tween 20 in water. The contents were mixed for 15 minutes and then centrifuged at 10,000 x g for 10 minutes. The supernatants were recovered and analyzed for protein and glycoproteins.

The percent removal of total protein and percent recovery of glycoproteins from aqueous extracts of liver tissue and the water insoluble fraction that was solubilized with Tween 20 are given in Table 5.

TABLE 5

| | % Total | % |
|---|---|---|
| Sample | Protein Removal | Glycoprotein Recovered |
| Water soluble liver fraction | 78.6 | 74.2 |
| Detergent solubilized | 57.72 | 48.21 |
| fraction | | |

The volumes obtained for protein recovery and glycoproteins recovery from the CPPA treated water soluble fraction from liver homogenates are comparable to those obtained with plasma (see Reference Examples 3 and 4). The efficiency of protein removal and glycoprotein recovery of CPPA treated surfactant solubilized tissue was less than that reported for soluble fraction.

Reference Example 6. Recovery of Glycoprotein Enzymatic Activity.

In this Example the oxidoreductase activity of the glycoprotein enzyme horse radish peroxidase (HRP) dosed into plasma and then treated with CPPA was determined.

The enzymatic assay procedure for horseradish peroxidase and the reagent 2,2"azino-bis (3-ethylbenzthiazoline-6-sulfonic acid (ABTS)) as substrate were obtained from Sigma Chemical Co. (St. Louis, MO). The assay was conducted at 25 ºC, pH = 5.0.

The method utilizes a continuous spectrophotometric rate determination. The change in absorbance after a two-minute period of reaction represents the enzyme activity.

100uL of a horseradish peroxidase solution containing 760 units of enzyme activity was added to 0.9mL of sheep plasma. 1 mL of CPPA suspension was then added. The tube was mixed by repeated inversions for 15 minutes and then centrifuged at 10,000 x g for 10 minutes. The supernatant was recovered and the volume was determined to be 1.8mL. The control consisted of 100ul of the same peroxidase solution added to 0.9mL of sheep plasma and then adjusted to 1.8mL with phosphate buffered saline pH 7.3.

100uL and 10uL volumes of the CPPA treated peroxidase containing plasma as well as 100uL and 10uL of the control plasma, respectively, were reacted with the substrate and the change in absorbance was recorded.

The recovery of peroxidase activity from plasma dosed with HRP and treated with CPPA is given in Table 6.

TABLE 6

| | | | % total peroxidase |
|---|---|---|---|
| Samples | Volume Tested (ul) | ΔA₄₀₅ | activity recovered |
| Plasma-HRP | 100 | 2.8949 | 100 |
| | 10 | 0.7556 | 100 |
| Plasma-HRP | 100 | 2.7315 | 94.36 |
| treated with CPPA | 10 | 0.6366 | 84.25 |

The reaction kinetics of the CPPA treated and untreated peroxidase containing plasmas were identical. Based on the assumption that the untreated dosed plasma maintained 100% of the initial enzyme activity, the recovery of peroxidase activity from the CPPA treated samples ranged from 84% to over 94%.

Reference Example 7. Protein Resolution on Crosslinked Chitosan Beads (from US Pat. 5,770,712)

An alternative mode of protein isolation is selective and is geared towards the isolation of single protein species. The method utilizes a large diameter low-density chitosan hydrogel construct (US Patent No. 5,770,712) that contains bound charged groups (anion or cation) or ligands cross-linked to the beads; This construct or chitosan support material is made with crosslinking and installation of a spacer arm between the chitosan matrix and an epoxy terminal group at the distal end of the spacer arm (US Pat. 5,770,712).

Salt elution studies were conducted to compare the binding capacity and binding selectivities of DEA-activated (with spacer) crosslinked chitosan beads, designated "B1" versus DEAE-activated (without spacer), crosslinked chitosan beads, designated "CB1". The beads were loaded into a 1.5 cm diameter x 13 cm bed height column was used for the elution experiments. The load/wash buffer was 20 mM potassium phosphate dibasic at a pH of 7.5. 100 mg bovine serum albumin (BSA; Sigma Chemical) was diluted to a final BSA concentration of 2.0 mg BSA/mL.

Different elution buffers based on 20 mM potassium phosphate dibasic at a pH of 7.5 combined with 25 mM to 200 mM of 1.0 M NaCl, in the increments indicated in Table 1. The load/wash flow rate was 5.0 mL/min and the elution flow rate was 10.0 ml/min. The elution absorption of the BSA solutions were taken after elution in the column and compared to the initial BSA reading. The BSA sorption in the beads was determined by difference from the initial load.

The overall test results are summarized in Table 7 below.

TABLE 7
BSA Eluted at Salt Concentration (% of Load)

| Activation Bead Type | Flow through/Wash | 25 mM | 50 mM | 75 mM | 100 mM | 200 mM | 1.0 M |
|---|---|---|---|---|---|---|---|
| CB1 (DEAE no spacer) | 21 | 7 | 22 | 17 | 15 | 20 | 2 |
| B1 (spacer-DEA) | 38 | 21 | 17 | 5 | 3 | 3 | 2 |

Ft = Flow through

The results show that the reference ion exchanger beads CB1 exhibited a high affinity for the test protein, BSA (bovine albumin serum); however, the results also revealed that the BSA partially eluted over a relatively broad range of salt concentrations from 25 mM to 200 mM. This broad elution behavior suggests that a wide range of binding affinity exists between the protein molecules and the ion exchange groups on the crosslinked chitosan beads lacking a spacer arm. It is further concluded that non-selective binding was reduced by installing a spacer arm between the chitosan matrix and terminal anion exchange group, as demonstrated by the results achieved for the beads B1. Namely, the beads B1 demonstrate markedly improved selectivity of binding, as most of the BSA elutes in the 25 mM to 50 mM NaCl concentration range.

Reference Example 8. Binding and Elution of Two Proteins Using B1 Beads.

B1 beads containing the positively charged ligand:

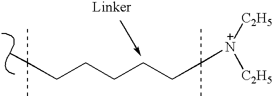

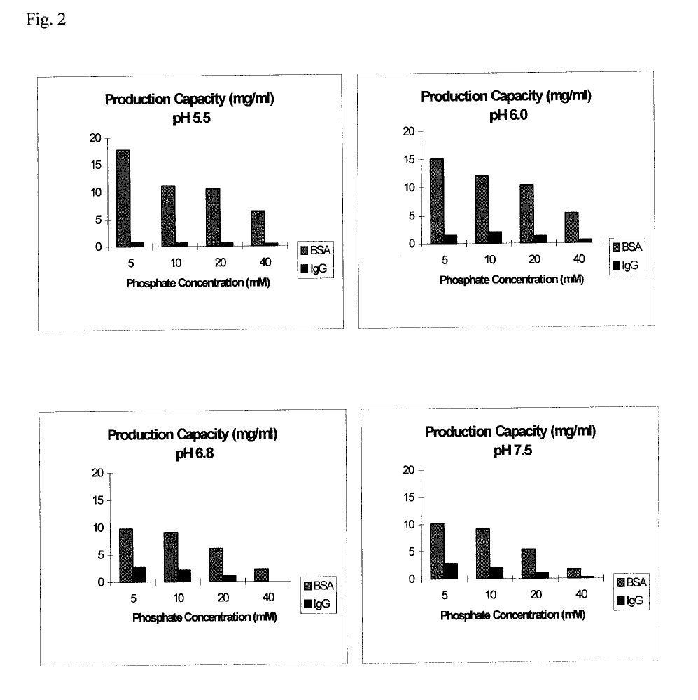

were used to study the complex formation with albumin and IgG. The results are reported in Fig. 2, wherein

Production Capacity = Elution Capacity at >90% protein yield.

Albumin, an acidic protein, bound at higher capacity at low pH (5.5) than at neutral pHs (Fig. 2). This suggests that in addition to simple electrostatic interactions based on the amphoteric character of the protein, other secondary interactions are participating in binding including hydrophobic and partial ionic interactions.

Example 1. Selectivity of Protein Resolution in a Goat Serum.

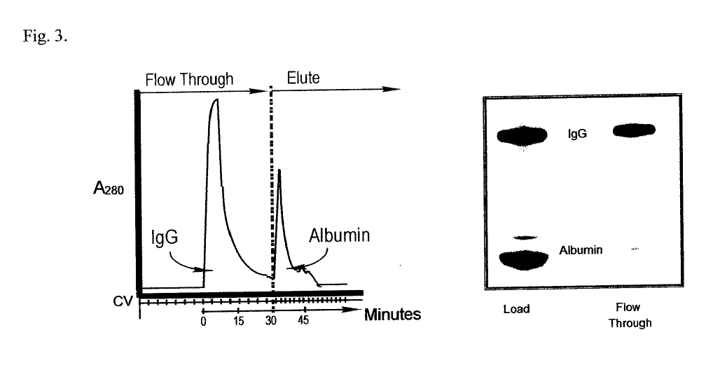

Using the same support B1as in Reference Example 7, selectivity of protein resolution was examined using a complex mixture, goat serum, as the sample. The results, shown in Fig. 3, illustrate the effective purification of polyclonal antibodies and the removal of peptides and proteins (A₂₈₀) from hyper-immune goat serum at 6 minute load cycle Residence Time.

The results show that IgG flows through, and albumin and other serum components bind with a high degree of selectivity. Albumin can be recovered by changing the composition of the elution buffer. This result indicates that low molecular weight ligands can be highly selective. Without wishing to be bound by theory or hypothesis, it is believed that a factor contributing to the selectivity is a low affinity of the ligand for the protein solute.

Example 2. Selectivity of Chitosan derivatized with a Carboxylate Ligand.

Crosslinked chitosan-spacer hydrogel was derivatized with propionate to provide a carboxylate ligand (Chitosan-COO):

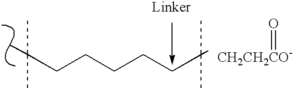

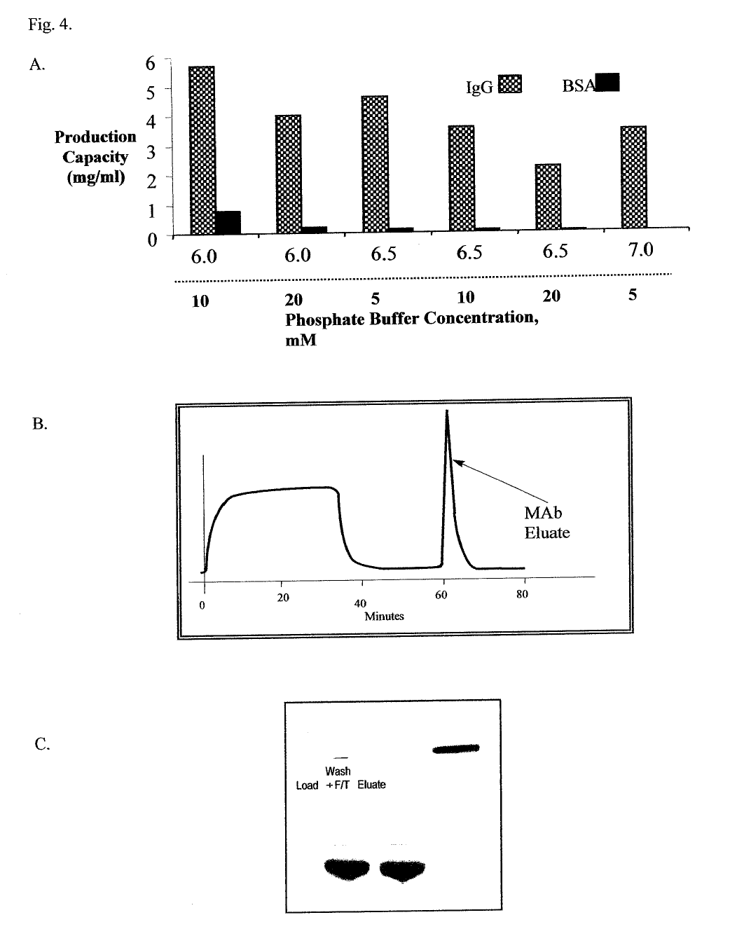

This composition displayed opposing binding characteristics to those of chitosan-DEA (Example 1), passing much protein material and retaining the antibody materials, as shown in Fig. 4.

Example 3. A Plate-Based Protein Fingerprint Characterization

In this Example resolution of three very closely related proteins, bovine serum albumin, glycated bovine serum albumin (chemically modified to contain 1-2 moles of hexose per mole of albumin), and rabbit serum albumin using chitosan-COO was tested in a simulated microtiter plate based format. 2 mL microtubes were used for these studies as a model for high throughput applications using multiwell plates. These beads are sufficiently large that they do not get transferred during pipetting. This aspect of the protocol will allow for straightforward adaptation to automated pipetting instruments (i.e. Tecan, Packard, Gilson). Smaller volumes and fewer beads are envisioned in the microtiter format.

The basic procedure was:

Equilibrate chitosan-COO beads to three pH conditions, 5.5, 6.3, and 7.1, by successive washes of low molarity buffers to the required pH.

Add settled beads to each tube plus additional buffer.

Apply protein sample each sample had a concentration of approximately 30 mg/mL of albumin. Mix for 10 minutes.

Wash with buffer. Mix for 10 minutes.

Desorb with pH 8, containing 250 mM NaCl buffer. Mix for 10 minutes.

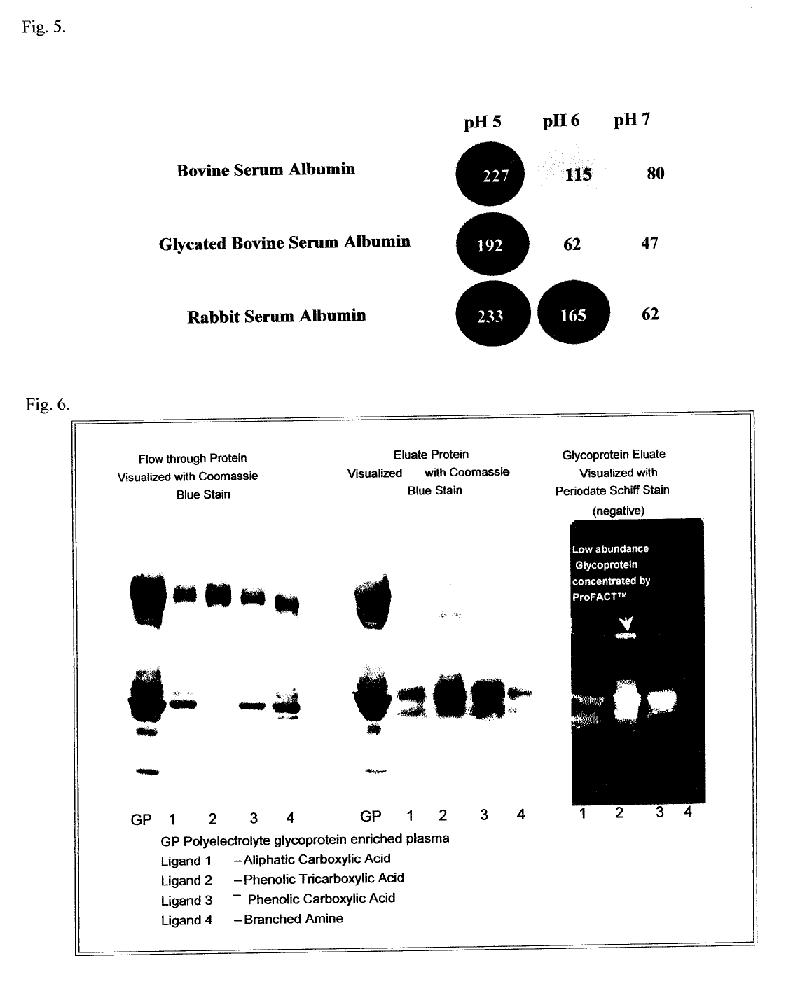

A colorimetric protein assay was performed on each of the wells and analyzed by spectrophotometry. The results are shown as relative "scores" in Fig. 5. It is seen that even with a small degree of carbohydrate substitution (glycated BSA), the binding affinities on chemically modified beads are very distinguishable from the unmodified protein. In addition, and even species-dependent amino acid differences between the closely related bovine and rabbit albumins affect their relative binding affinities. Applicants believe these results are unexpected by a worker of ordinary skill in the art. The matrix of scores for a particular protein under varying conditions may be interpreted as a "fingerprint" of that protein.

The results in Examples 2 and 3 demonstrate that beads comprising selectivity-determining ligands of the present invention can be adapted to a microtiter based, bind/elute protocol, and that by using such a format, unique binding affinity fingerprints are possible. By extension, non-binding affinity fingerprints are suggested as well.

Thus, with chemically modified chitosan beads, resolution previously achievable only by the use of bio-ligand affinity (e.g., Protein A), along with the ability to fingerprint closely related proteins is now obtainable with commercially available, non-proprietary, low molecular weight ligands (see U. S. Pats. 5,294,681 and 5,453,493). In contrast to the development of classical affinity ligands, these ligands were not designed for a specific purification task nor required any prior knowledge of the protein structure or function. Instead, they were characterized through a methodical process that can be applied across thousands of simple chemical substrates that are commercially available. In this way future ligands will be systematically characterized so as to progressively refine the protein sorting process.

Example 4. Two-Step Glycoprotein Resolution on Chitosan-DEA Beads

The results given in Reference Examples 3 and 4 have shown that treatment of serum or plasma with polyelectrolyte CPPA removes most of the albumin and some of the immunoglobulins, enriching glycoproteins in the supernatant fraction. Consequently, use of CPPA enrichment as a first step in the analysis of glycoproteins is recommended to reduce the complexity of a protein mixture applied to a specificity-determining substrate. Furthermore, the remaining glycosylated fraction is in its native state and biologically active (see Example 11).

This Example demonstrates that glycoproteins may be first enriched from serum, and then can be applied to a specificity-determining substrate (chitosan-DEA in bead format) for further resolution and analysis tested in a well based format.

The serum was treated with the polyelectrolyte CPPA as previously described, and the supernatant thus obtained, enriched for glycoproteins, was applied to the chitosan-DEA beads, and equilibrated to pH 5.5. Approximately 60% of the serum glycoproteins were captured and eluted from the beads. A visual inspection of an SDS-PAGE electrophoretogram showed that virtually all of the immunoglobulins did not bind. Therefore, these results suggest that the chitosan-DEA beads substantially differentiate immunoglobulins from a large majority of glycoproteins from serum.

To further assess the proteomic capability of the chitosan-DEA beads, a model glycoprotein, alpha-1-acid-glycoprotein, was studied. A binding study demonstrated that a pure isolate of alpha-1-acid-glycoprotein bound with high efficiency to the chitosan-DEA beads (data not shown). Alpha-1-acid-glycoprotein was dosed into serum (1 mg/mL final concentration), and compared to a control undosed serum. Each of the serums were first treated with CPPA adsorbent, and then subsequently applied to the chitosan-DEA beads.

Clear differentiation of the dosed vs. the undosed sample can be seen from the carbohydrate analysis for both the CPPA treated samples (stated as Enriched Glycoprotein Fraction), and the subsequent application to chitosan-DEA beads (Table 8).

TABLE 8

| | Serum | Dosed | % |
|---|---|---|---|
| | ControlMeasurement | SerumMeasurement | Increase |
| Enriched | 1.06 | 1.25 | 18 |
| Glycoprotein | | | |
| Fraction | | | |
| Eluate from | 0.80 | 0.92 | 15 |
| chitosan-DEA | | | |
| Beads | | | |

This Example demonstrates that combined application of glycoprotein enrichment with polyelectrolyte CPPA and chitosan-DEA, applied to a very crude sample (serum) resolves proteins of varying character. Such capabilities are necessary in high throughput proteomics analyses.

Example 5. Crosslinked Chitosan Preparations Bearing Different Specificity-Determining Ligands

The general procedure for chitosan cross linking and installation of an epoxy terminal linking group is described in US Patent Number 5,770,712, incorporated here by reference. The procedure was repeated to prepare 14 samples, each with a different specificity-determining ligand.

Crosslinking of Chitosan Beads

300mL untreated chitosan beads were first washed with water. The beads were then washed several times with 50% isopropyl alcohol. The beads were further washed with 100% isopropyl alcohol five to six times. Then 150mL of 1,4-butanediol diglycidyl ether was added to 600mL of isopropyl alcohol in a reaction flask plus 300mL of chitosan beads. The reaction was stirred for 24 hours at a temp of 55 deg C. The beads were then washed with isopropyl alcohol several times for further processing.

Fourteen ligands were immobilized on cross linked chitosan (See Table 9).

Ligand Attachment via Epoxy Coupling Using Ligands A through K

10 grams quantities of cross linked epoxy derivatized beads were suspended in 50 mL

TABLE 9

| | | | |
|---|---|---|---|
| Letter | | | |
| Desig- | | | Coupling |
| nation | Name | Formula | Chemistry |
| A | 2-Butylaminoethanol | CH_{3(CH}₂₎_{3NHCH}_{2CH}_{2OH} | Epoxy |
| B | tert-Butylamine | (CH₃₎_{3CNH}₂ | Epoxy |
| C | 2-(tert-Butylamino)- | (CH₃₎_{3CNHCH}_{2CH}_{2OH} | Epoxy |
| | ethanol | | |
| D | 2,4,6 Tris- | [(CH₃₎_{2NCH}_{2]}_{3C}_{6H}_{2OH} | Epoxy |
| | (dimethylamino- | | |
| | methyl)Phenol | | |
| E | Octylamine | CH_{3(CH}₂₎_{7NH}₂ | Epoxy |
| F | Diisopropylamine | [(CH₃₎_{2CH]}_{2NH} | Epoxy |
| I | n Hexylamine | C_{6H}_{13NH}₂ | Epoxy |
| J | Diethylamine | (C_{2H}₅₎_{2NH} | Epoxy |
| K | Benzylamine | C_{7H}_{9N} | Epoxy |
| 1 | 1,2,4,5 Benzenetetra | | Amino |
| | carboxylic dianhydride | | |
| 2 | Trimethylacetic | [(CH₃₎_{3CCO]}_{2O} | Amino |
| | anhydride | | |
| 4 | Benzoic anhydride | C_{14H}_{10O}₃ | Amino |
| 5 | Succinic anhydride | C_{4H}_{4O}₃ | Amino |
| 6 | Diphenyl Maleic | C_{16H}_{10O}₃ | Amino |
| | Anhydride | | |

aliquots of isopropyl alcohol and reacted with 20 times excess (0.01 mole) amounts of ligands A through K. Reactions were conducted in an orbital shaker for 24 hours at room temperature. The individual batches of reacted beads were then washed and suspended in isopropyl alcohol and stored at 4 deg C.

Preparation of Amino derivatized Crosslinked Chitosan Ligands

50 grams of epoxy derivatized beads were added to 200mL of a 10% aqueous ammonia solution. The mixture was shaken overnight in an orbital shaker at room temperature. The beads were then washed with water and then washed and suspended in isopropyl alcohol.

Ligand Attachment via an Amino group -- Ligands 1, 2, and 4-6

10 grams of amino derivatized crosslinked chitosan was washed with acetone and reacted with 20 times excess (0.01 mole) amounts of ligands 1, 2, 4, 5, and 6. The mixtures were shaken as previously described. The reacted beads were then washed with isopropyl alcohol for further processing.

Example 6. Preparation and use of additional ligands derivatized to chitosan

300mL untreated chitosan beads are first washed with water. The beads are then washed several times with 50% isopropyl alcohol. The beads are further washed with 100% isopropyl alcohol five to six times. Then 150mL of 1,4-butanediol diglycidyl ether is added to 600mL of isopropyl alcohol in a reaction flask plus 300mL of chitosan beads. The reaction is stirred for 24 hours at a temp of 50-60 deg C. The beads are then washed with isopropyl alcohol several times for further processing.

Derivatization using spacers ending in epoxide groups

10 grams of cross linked epoxide-spacer derivatized beads are suspended in 50 mL aliquots of isopropyl alcohol and reacted with 20 times excess (0.01 mole) amounts of a ligand shown in Table 10 suitably derivatized to react with epoxide groups. Reactions are conducted in an orbital shaker for 24 hours at room temperature. The individual batches of reacted beads are then washed and suspended in isopropyl alcohol and stored at 4 deg C.

TABLE 10

| | |
|---|---|
| | Model Target |
| Ligand Chemistry | Protein(s) |
| 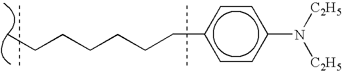 | Albumin |
| 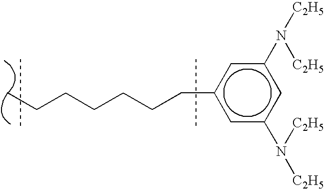 | Transferrin, Alpha Feto Protein |
| 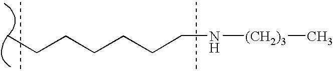 | Ceruloplasmin |
| 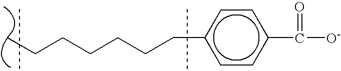 | Human Chorionic Gonadotropin (HCG) |
| 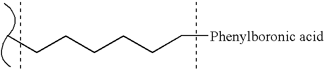 | Glycoproteins |
| 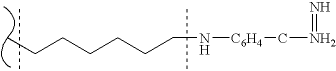 | Serine Proteases |

Preparation of Amino derivatized Crosslinked Chitosan Ligands 50 grams of epoxide-spacer derivatized beads are added to 200mL of a 10% aqueous ammonia solution. The mixture is shaken overnight in an orbital shaker at room temperature. The beads are then washed with water and then washed and suspended in isopropyl alcohol.

Ligand Attachment via an Amino group

10 grams of amino derivatized crosslinked chitosan as prepared in the preceding paragraph is washed with acetone and reacted with 20 times excess (0.01 mole) amounts of a ligand chosen from Table 10 suitably derivatized to react with a primary amine group. The mixtures are shaken as previously described. The reacted beads are then washed with isopropyl alcohol for further processing.

Example 7. Preferential Binding Character of Ligands attached to derivatized beads

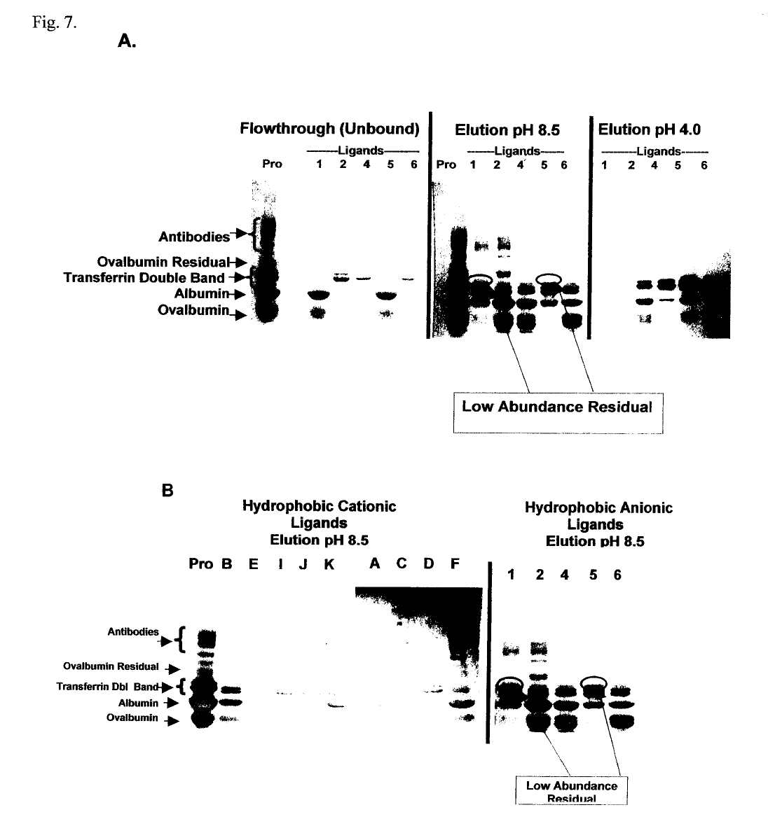

Four derivatized crosslinked chitosan preparations, bearing, respectively, an aliphatic carboxylic acid, a phenolic tricarboxylic acid, a phenolic carboxylic acid, and a branched amine, were tested for their ability to resolve protein in plasma. The results are shown in Fig. 6. These electrophoretic profiles show that certain proteins flow through, while others are preferentially retained to different extents by macrobeads with the four different ligands. These results show that differing ligand binding affinities sort proteins in differential fashion, and can reveal low abundance proteins in a mixture. This selectivity contributes to the analysis of proteins in proteomics investigations.

Example 8. Evaluation of Ligand Substituted Crosslinked Chitosan Beads

The ability of crosslinked chitosan derivatized with various ligands to resolve proteins in a mixture was examined in this Example. Transferrin, bovine serum albumin, and ovalbumin (Sigma Chemical Co., St. Louis, MO) were mixed together for this study.

250uL of each preparation of chitosan-ligand derivative beads were put in a 1.5mL tube and the excess buffer removed. To the beads, 250uL of the protein mixture was added and the samples were then mixed on a shaker for 15-20 minutes. The supernatant was then recovered.

The beads were then washed with 1mL of 10mM Tris buffer pH 6.0 and the washings were discarded. The beads left were then eluted off using 250uL of 100mM Tris buffer pH 8.5 and mixed for 5 minutes. The second supernatant was recovered. In addition the remaining beads were also washed with 0.4mL of 100mM potassium acetate buffer pH 4.0 and mixed for 15 minutes. Again the supernatants were withdrawn and retained for further analysis.

The supernatants were subjected to SDS-PAGE on a 12% gel. The resulting electrophoretograms are shown in Fig. 7.

It is seen that the fingerprints of the proteins recovered from the beads derivatized with the different ligands are dramatically different. In addition to the different protein profiles exhibited by the unbound (first) supernatant, there were also marked differences in elution profiles using different pH values. Resolution of the protein mixtures with ligand 1 and 5 uncovered a low abundance residual band. These results show that specificity-determining supports of the present invention, employed at differing ambient conditions, have the ability to resolve complex protein mixtures under batch processing methodology.

Example 9. Selection and Resolution of Glycoproteins from Plasma

In this Example sheep plasma was treated first with acidic polyelectrolyte hydrogel to separate a glycoprotein fraction. The glycoproteins were resolved using various chitosan-ligand bead preparations in a batch (microtiter plate) format.

1.0 mL of Sheep Plasma (Lot# 109205PNaEDTAI) was added to 1.0mL acidic polyelectrolyte hydrogel, and shaken for 15 minutes using an orbital shaker. After centrifuging the supernatant was retained. This first supernatant fraction was then subjected to a set of chitosan-ligand derivatized beads. The beads were first washed with 10mM Potassium Phosphate buffer pH 6.0 until the pH was even across all bead samples. The supernatant fraction from the previous step was then diluted to 5mL using distilled water.

0.4mL of the diluted sample was then added to 0.25mL of derivatized bead sample in a 96-well receiver plate. The plate containing the mixture was then mixed for 10 minutes and sets of second supernatants were recovered and retained.

The derivatized beads were then washed using 1.0mL of 10mM Potassium Phosphate buffer pH 6.0 for five minutes using a shaker. The washings were then discarded. 0.4mL of a buffer with a higher pH, 100mM Tris buffer pH 8.5, was added to the beads and mixed for 15 minutes using a shaker. The supernatant was withdrawn and retained for further analysis. In addition the remaining beads were also washed with 0.4mL of 100mM Potassium Acetate buffer pH 4.0 and mixed for 15 minutes. Again the supernatants were withdrawn and retained for further analysis.

The supernatants from all of the samples above were examined by 12% SDS polyacrylamide electrophoresis and stained for protein using Coomassie Blue and for glycoprotein using the Pierce Chemical Co. Glycoprotein detection kit.

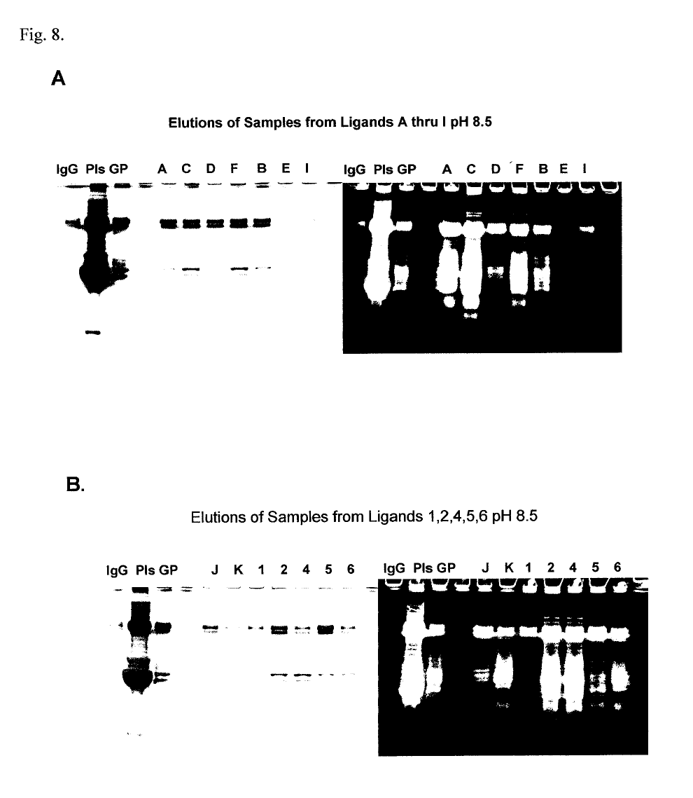

As may be seen in the results shown in Fig. 8, there are marked differences in the fingerprints of the glycoproteins recovered from the different ligands employed.

Example 10. Analysis Of Alpha-1 Acid Glycoprotein Dosed Into Plasma Using Glycoprotein Enrichment And Chitosan-Ligand Beads.

The chitosan-ligand beads were equilibrated by first washing with 10mM Potassium Phosphate buffer pH 6.0 until the pH was even across all bead samples. 1.0 mL of Sheep Plasma (Lot# 109205PNaEDTAI) dosed with 1mg/mL alpha-1 acid glycoprotein was added to 1.0mL acidic polyelectrolyte hydrogel, the mixture was then mixed for 15 minutes using an orbital shaker. The mixture was then centrifuged and the supernatant was retained. The supernatant was then subjected to a set of chitosan-ligand derivatized beads (C, D, E, J, 1 and 6 (Table 9)). The supernatant (flowthrough) from the previous step was then diluted to 5mL using distilled water.

0.4mL of Sample (Diluted Flowthrough) was then added to 0.25mL of chitosan bead samples in a 96-well microtiter plate. The plate containing the mixtures was then mixed for 10 minutes and the second supernatant was recovered and retained.

The beads were then washed using 1.0mL of 10mM Potassium Phosphate buffer pH 6.0 for five minutes using a shaker. The washings were then discarded. Further, 0.4mL of 100mM Tris buffer pH 8.5 was added to the beads and mixed for 15 minutes using a shaker. The supernatant was withdrawn and retained for further analysis. In addition the remaining beads were also washed with 0.4mL of 100mM Potassium Acetate buffer pH 4.0 and mixed for 15 minutes. Again the supernatants were withdrawn and retained for further analysis.

Eluted fractions were analyzed by spectrophotometrically OD_{280.} The results are presented in Table 11. A unique profile is particularly evident with Ligand J where dosed Alpha-1 acid glycoprotein is eluted.

TABLE 11
Initial Profile Analyses of Control Plasma vs.Dosed
(1 mg/mL a1-Acid Glycoprotein) Plasma

| | Serum Control | |
|---|---|---|
| | OD280 × 1000 | Dosed Serum OD280 × 1000 |
| Eluate from Chitosan C | 683 | 630 |
| Eluate from Chitosan C | 197 | 95 |
| Eluate from Chitosan E | 89 | 126 |
| Eluate from Chitosan J | 45 | 193 |
| Eluate from Chitosan 1 | 204 | 212 |
| Eluate from Chitosan 6 | 1127 | 1087 |

Example 11. Analysis Of Horse Radish Peroxidase Dosed Into Plasma Using Glycoprotein Enrichment And Chitosan-Ligand Beads.

This Example presents a determination of the oxidoreductase activity of the glycoprotein enzyme, horseradish peroxidase (HRP), dosed into plasma at a level of 2 mg HRP to 1 ml sheep plasma. The dosed plasma was first enriched for glycoproteins by an CPPA, and then applied to the specificity-determining substrate for resolution and/or purification. The assay for HRP activity is the same as described in Reference Example 6.

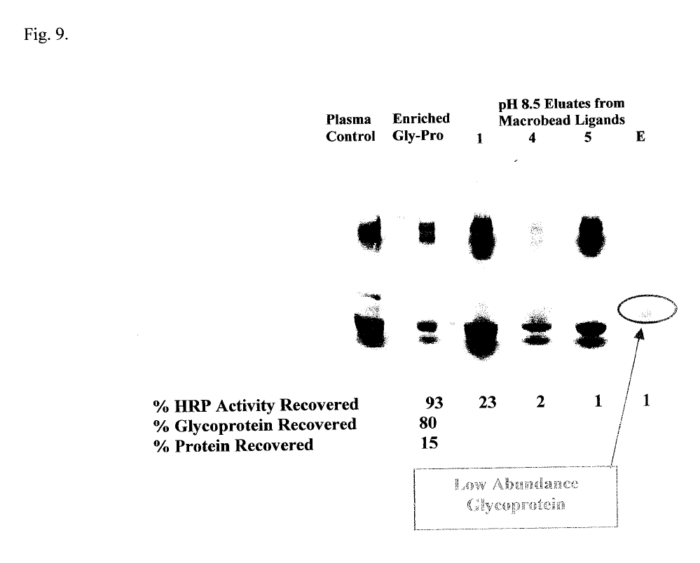

Results obtained for four ligands bound to a chitosan specificity-determining substrate are shown in Fig. 9. Ligands 1,4,5 are hydrophobic anionic and Ligand E is hydrophobic cationic (see Example 5).

The HRP activity was 93% retained after enrichment by CPPA, demonstrating the efficiency of activity recovery possible through adsorbent treatment. The selective occurrence of HRP activity from Ligand 1 of the 4 ligands tested, illustrates the ability to trace and sort enzyme sub-populations using the two-step technologies for glycoprotein sorting and functional analysis. Particularly noteworthy again is the low abundance glycoprotein (carbohydrate stain not shown) "uncovered" and isolated to virtually single component purity by the combined two-step resolution technologies.

These results show that, using spiking experiments in plasma, ligand differentiation of the glycoprotein HRP has been detected at ng levels. This demonstrates that ligand differentiation and functional integrity of the sorting process extends to very low abundance levels (million-fold minimum), a critical need in proteomic discovery analysis.

Example 12. Proteomics Investigation Of Glycoproteins Present In A Biological Sample.

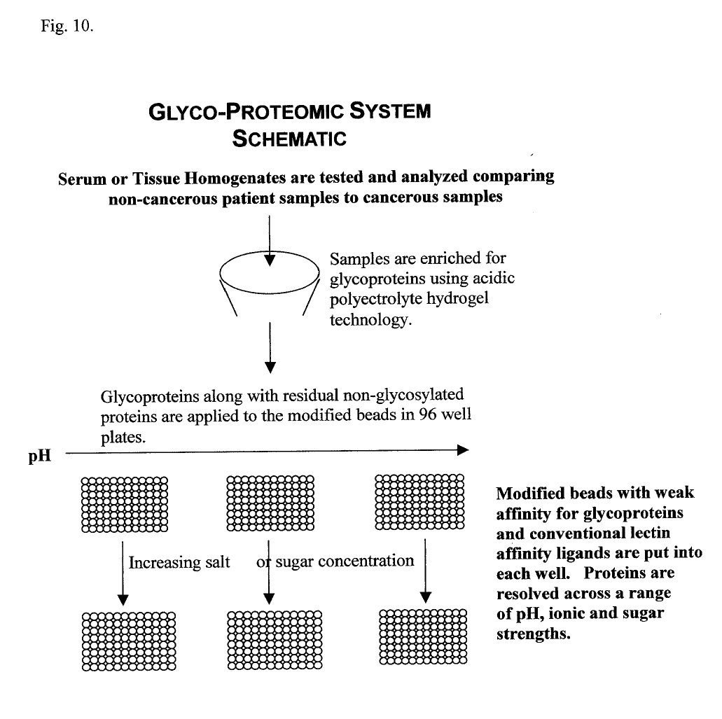

Fig. 10 shows a schematic representation of a comprehensive assay conducted on a biological sample, directed at glycoproteins. First, glycoproteins are enriched from the sample by treatment with an acidic polyelectrolyte hydrogel, remaining in the supernatant fraction. Then, samples of the supernatant are arrayed into microtiter plates containing one or more chitosan-ligand derivatized beads, in conjunction with an optional use of a lectin-derivatized support. Treatment of the wells with ambient fluid conditions having varying pH, ionic composition and/or saccharide content elutes various glycoproteins from the wells in a condition-dependent fashion. These are assayed by a variety of physical and functional assays to characterize the properties of each well.

At the end of the elution cycles, each well will contain differentiated protein(s) in native, biologically functional states. The protein from the modified beads are digitally and functionally analyzed. Reporter/probe measurements for protein, carbohydrate and biological activity (i.e. kinase activity, or more specific ELISA's) effectively score each well in a precise digital format. Each type of detection adds further granularity to the discovery process and is strategically selected for each situation.

At the end of the resolution and detection process, each individual well has a particular signature denoted by its protein, carbohydrate and bioassay readings. Collectively the wells configure a digital signature pattern of all the proteins in the sample. Through comparison algorithms, including those based on neural networks, these protein signature patterns are assessed for quantifiable differences from control samples. Control samples are normal tissue sources or pathological (cancer or other disease) samples. Once it is established that for any given well or group of wells, differences are beyond a normal experimental variance, then validated drug targets have been discovered.

At the discretion of the investigating scientist, supplementary differentiation and analysis by capillary electrophoresis, additional biological activity assays, amino acid fingerprinting by mass spectrometry, or crystallography are performed for further characterization of resolved targets of interest.

What is Claimed is:

1. A specificity-determining substrate that forms a complex with a protein molecule in a homogenous fashion, wherein the specificity-determining substrate comprises a specificity-determining ligand bound to a support, wherein optionally the substrate further comprises a spacer bound between the ligand and the support, wherein the spatial separation between adjacent ligand groups is greater than a predetermined minimum distance, and provided that the substrate is other than a crosslinked chitosan bearing a diethylamino or diethylaminoethyl ligand.

2. The substrate described in claim 1 wherein the specificity-determining ligand is chosen from the group consisting of a monosaccharide, a disaccharide, a trisaccharide, an oligosaccharide,

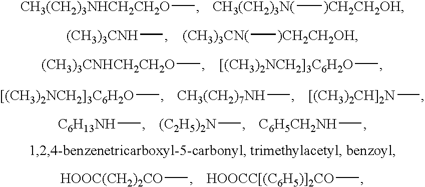

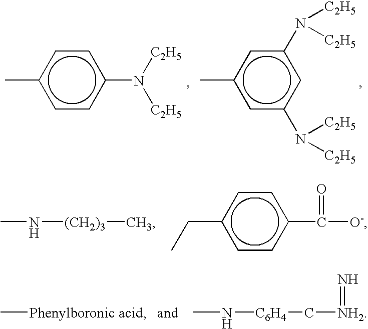

3. The substrate described in claim 1 wherein the specificity-determining ligand comprises 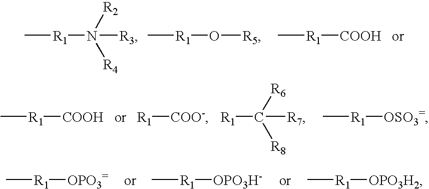
wherein R₁ is chosen from the group consisting of a normal or branched aliphatic moiety, a cycloalkyl moiety, an aromatic moiety, an aralkyl moiety, an aliphatic-aliphatic ether, an aliphatic-aromatic ether, an aliphatic moiety having a secondary or tertiary alcohol, a phenolic moiety, an aliphatic moiety having a secondary or tertiary amine, an aniline moiety, an aliphatic carboxyl, sulfate, sulfonate, or phosphate ester, an aromatic carboxyl, sulfate, sulfonate or phosphate ester, and an aromatic heterocycle;
and wherein R₂, R₃, R₄, R₅, R₆, R₇, and R₈, are independently chosen from the group consisting of hydrogen, a normal or branched aliphatic radical, a cycloalkyl radical, an aromatic radical, an aralkyl radical, an aliphatic-aliphatic ether, an aliphatic-aromatic ether, an aliphatic radical having a primary, secondary or tertiary alcohol, a phenolic radical, an aliphatic radical having a primary, secondary or tertiary amine, an aniline radical, an aliphatic carboxylate, sulfate, sulfonate, or phosphate group, an aromatic carboxylate, sulfate, sulfonate or phosphate group, and an aromatic heterocycle.

4. The substrate described in claim 1 wherein a spacer is 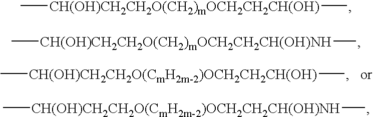
where m is an integer between 2 and 10.

5. The substrate described in claim 1 wherein the support is chosen from the group consisting of a glass surface, a silica surface, a ceramic surface, a plastic surface, a resin particle, a bead, a gel, a polyelectrolyte, and a hydrogel.

6. The substrate described in claim 5 wherein the support is other than a surface and wherein the solids content of the support when equilibrated with an ambient fluid is less than a predetermined maximum content.

7. The specificity-determining substrate described in claim 6 wherein the solids content of the support is less than about 8% w/v.

8. The substrate described in claim 1wherein the support comprises a polysaccharide.

9. The substrate described in claim 1 wherein the support comprises chitosan.

10. A complex comprising a specificity-determining substrate described in claim 1and a protein molecule.

11. A complex comprising a specificity-determining substrate described in claim 2 and a protein molecule.

12. A complex comprising a specificity-determining substrate described in claim 3 and a protein molecule.

13. A complex comprising a specificity-determining substrate described in claim 4 and a protein molecule.

14. The complex described in claim 10 wherein the support is chosen from the group consisting of a glass surface, a silica surface, a ceramic surface, a plastic surface, a resin particle, a bead, a gel, a polyelectrolyte, and a hydrogel.

15. The complex described in claim 14 wherein the support is other than a surface and wherein the solids content of the support when equilibrated with an ambient fluid is less than a predetermined maximum content.

16. The complex described in claim 15 wherein the solids content of the support is less than about 8% w/v.

17. The complex described in claim 10 wherein the support comprises a polysaccharide.

18. The complex described in claim 10 wherein the support comprises chitosan.

19. An array comprising a plurality of loci wherein each locus comprises a specificity-determining substrate described in claim 1.

20. The array described in claim 19 wherein the specificity-determining ligand at a first locus differs from a specificity-determining ligand at a second locus.

21. The array described in claim 19 wherein the specificity-determining ligand at a first locus is identical to a specificity-determining ligand at a second locus.

22. An array comprising a plurality of loci wherein each locus comprises a specificity-determining substrate described in claim 2.

23. An array comprising a plurality of loci wherein each locus comprises a specificity-determining substrate described in claim 3.

24. An array comprising a plurality of loci wherein each locus comprises a specificity-determining substrate described in claim 4.

25. A method of resolving a first protein from a fluid comprising one or more species of native, biologically active protein molecules, wherein the first protein retains its native structure and its biological activity, the method comprising the steps of:
a) contacting the fluid with a specificity-determining substrate described in claim 1, thereby forming a complex comprising the first protein; and
b) separating the fluid so contacted from the complex;
thereby resolving the first protein from the fluid.

26. The method described in claim 25 wherein the fluid comprises a plurality of species of protein molecule, and the contacted and separated fluid comprises a second protein.

27. The method described in claim 25 wherein the specificity-determining ligand is chosen from the group consisting of a monosaccharide, a disaccharide, a trisaccharide, an oligosaccharide,

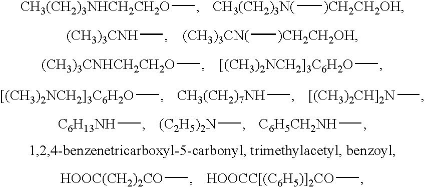

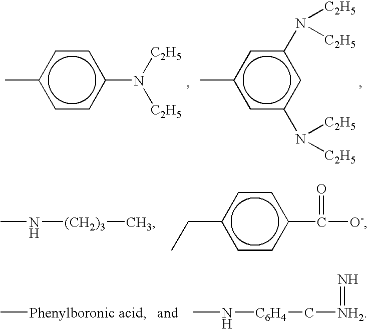

28. The method described in claim 25 wherein the specificity-determining ligand comprises 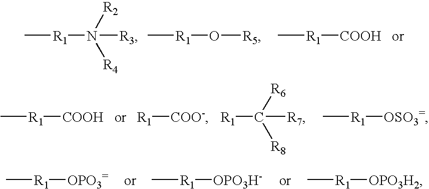
wherein R₁ is chosen from the group consisting of a normal or branched aliphatic moiety, a cycloalkyl moiety, an aromatic moiety, an aralkyl moiety, an aliphatic-aliphatic ether, an aliphatic-aromatic ether, an aliphatic moiety having a secondary or tertiary alcohol, a phenolic moiety, an aliphatic moiety having a secondary or tertiary amine, an aniline moiety, an aliphatic carboxyl, sulfate, sulfonate, or phosphate ester, an aromatic carboxyl, sulfate, sulfonate or phosphate ester, and an aromatic heterocycle;and
wherein R₂, R₃, R₄, R₅, R₆, R₇, and R₈, are independently chosen from the group consisting of hydrogen, a normal or branched aliphatic radical, a cycloalkyl radical, an aromatic radical, an aralkyl radical, an aliphatic-aliphatic ether, an aliphatic-aromatic ether, an aliphatic radical having a primary, secondary or tertiary alcohol, a phenolic radical, an aliphatic radical having a primary, secondary or tertiary amine, an aniline radical, an aliphatic carboxylate, sulfate, sulfonate, or phosphate group, an aromatic carboxylate, sulfate, sulfonate or phosphate group, and an aromatic heterocycle.

29. The method described in claim 25 wherein the support is chosen from the group consisting of a glass surface, a silica surface, a ceramic surface, a plastic surface, a resin particle, a bead, a gel, a polyelectrolyte, and a hydrogel

30. The method described in claim 25 wherein the support comprises a polysaccharide.

31. The method described in claim 25 wherein the support comprises chitosan.

32. The method described in claim 25 further wherein, prior to performing step a) the fluid is pretreated by a method comprising the steps of:
a') contacting the fluid with a hydrogel comprising a water insoluble cross-linked polyhydroxy polycarboxylic acid having at least two strands each having a strand skeleton of the formula: 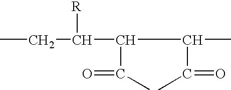
wherein R is H, OCH₃, or phenyl, and one carbonyl group of at least one maleoyl moiety thereof in each strand is covalently linked to a 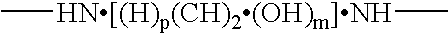
moiety to provide the presence therein of at least one cross linking moiety of the formula: 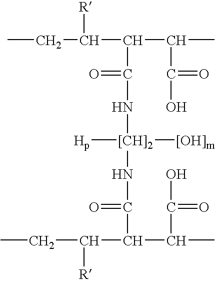
wherein R' is hydrogen or lower alkylene or lower alkoxy of 1-4 carbon atoms, or phenyl,
z is an integer of 1-4,
p is 0 or an integer up to z-1,
m is 1 or an integer up to z,
wherein the ratio of cross-links to poly (alkylene carbonic acid) strands is between about 1 and about 200 to 2,
to provide a hydrogel phase and a first supernatant; and
a") either
i) separating the hydrogel phase from the first supernatant and using the first supernatant as the fluid of step a) of claim 25, or
ii) separating the hydrogel phase from the first supernatant, then treating the hydrogel phase to release proteins adhering within it to provide a second supernatant comprising adhered proteins, and using the second supernatant as the fluid of step a) of claim 25.

33. A method of purifying one or more first proteins from a fluid comprising one or more species of native, biologically active protein molecules, wherein the purified first protein retains its native structure and its biological activity, the method comprising the sequential steps of
a) contacting the fluid with a specificity-determining substrate described in claim 1, thereby forming a complex described comprising the one or more first proteins
b) separating the contacted fluid from the complex; and
c) eluting the one or more first proteins from the specificity-determining substrate under conditions that retain the native structure and biological activity of the first protein
thereby providing one or more purified native, biologically active first proteins.

34. The method described in claim 33 wherein the fluid comprises a plurality of species of protein molecule, and the contacted and separated fluid comprises a second protein.

35. A method of characterizing one or more proteins in a fluid comprising one or more species of protein molecule, the method comprising the sequential steps of
a) providing a plurality of containers, wherein each container has a characteristic specificity-determining substrate described in claim 1and a characteristic set of ambient fluid conditions, and wherein ambient fluid conditions are described by one or more variables chosen from the group consisting of the temperature, the ionic strength, the fluid composition, an amount of a chaotropic agent, an amount of a detergent, an amount of an organic cosolvent, and the pH, wherein each of said ligand and said ambient fluid conditions in a first container may be the same or different from said ligand and said ambient fluid conditions in a second container
b) contacting the fluid with the plurality of containers, thereby promoting formation of a complex comprising the one or more proteins in a first container and inhibiting formation of a complex comprising the one or more proteins in a second container;
c) identifying the promotion of complex formation in the first container and the inhibition of complex formation in the second container; and
d) identifying the ligand and ambient fluid conditions in the first container and in the second container
thereby characterizing the one or more proteins.

36. A method of identifying one or more proteins in a sample fluid wherein the concentration of the one or more proteins in the sample fluid differs from the concentration of the one or more proteins in a reference fluid, the method comprising the sequential steps of
a) in a set of N containers, contacting the sample fluid with a specificity-determining substrate described in claim 1and an ambient fluid, wherein the ambient fluid has conditions described by one or more variables chosen from the group consisting of the temperature, the ionic strength, the fluid composition, an amount of a chaotropic agent, an amount of a detergent, an amount of an organic cosolvent, and the pH, wherein each container is characterized by a particular specificity-determining substrate and a particular fluid condition, the particular substrate and particular fluid conditions potentially promoting formation of a complex comprising the one or more proteins
b) determining the amount and/or species of protein molecules complexed with the substrate in each container of the sample fluid set
c) comparing the amount and/or species of protein molecules complexed with the substrate in each container of the sample set with the amount and/or species of protein molecules complexed with the substrate obtained by contacting a reference fluid with a specificity-determining substrate described in claim 1and an ambient fluid in a reference set of containers identical to the set used for the sample fluid, wherein the i-th container in the sample set and the i-th container in the reference set have the identical specificity-determining substrate and the identical ambient fluid conditions (1 ≤ i ≤N)
d) identifying an n-th container in the sample fluid set whose amount of one or more proteins complexed with the substrate differs from the amount of the one or more proteins complexed with the substrate in the n-th container in the reference fluid set (1 ≤ n ≤N); and
e) identifying the one or more proteins as the species whose amounts complexed with the substrate differ between the n-th sample container and the n-th reference container.
